(19) United States
(12) Patent Application Publication
(10) Pub. No.: US 2025/0099743 A1
(43) Pub. Date: Mar. 27, 2025

(54) MEDICAL DEVICE FOR TREATING DECOMPENSATED HEART FAILURE

(71) Applicant: Boston Scientific Scimed, Inc., Maple Grove, MN (US)

(72) Inventors: Ephraim Israel Ben-Abraham, Rochester, MN (US); Aaron Chalekian, Minneapolis, MN (US); Victor Kotov, St. Louis Park, MN (US)

(73) Assignee: Boston Scientific Scimed, Inc., Maple Grove, MN (US)

(21) Appl. No.: 18/894,729

(22) Filed: Sep. 24, 2024

Related U.S. Application Data
(60) Provisional application No. 63/542,908, filed on Oct. 6, 2023.
(60) Provisional application No. 63/586,982, filed on Sep. 29, 2023.
(60) Provisional application No. 63/540,617, filed on Sep. 26, 2023.

(51) Int. Cl. A61M 60/531; A61M 60/523; A61M 60/80

(52) U.S. Cl. CPC A61M 60/531; A61M 60/523; A61M 60/80; A61M 2205/04; A61M 2205/10; A61M 2205/3303; A61M 2205/3327; A61M 2205/3331; A61M 2205/502; A61M 2210/12; A61M 2230/005; A61M 2230/30

(57) ABSTRACT

This disclosure provides design, material, manufacturing method, and use alternatives for medical devices. An example medical device system for treating a heart, includes a control system including a processor and a pump, a hub coupled to the control system, a control system including a processor and a pump, a hub coupled to the control system, a first catheter shaft having a first lumen and a first end coupled to the hub, a second catheter shaft extending within the first lumen and having a first end couple to the hub, a first expandable member disposed on the first catheter shaft, wherein the first expandable member is configured to be positioned in the superior vena cava and a second expandable member disposed on the second catheter shaft distal to the first expandable member, wherein the second expandable member is configured to translate relative to the first expandable member.

### CROSS-REFERENCE TO RELATED APPLICATIONS

This application claims the benefit of priority under 35 U.S.C. § 119 of U.S. Provisional Application Number's 63/542,908, filed Oct. 6, 2023, 63/586,982, filed Sep. 29, 2023 & 63/540,617, filed Sep. 26, 2023, the entire disclosures of which is hereby incorporated by reference.

### TECHNICAL FIELD

The present disclosure pertains to medical devices, and methods for manufacturing medical devices. More particularly, the present disclosure pertains to medical devices including expandable members and pressure sensing devices connected with other structures, and methods for manufacturing and using such devices.

### BACKGROUND

A wide variety of intracorporeal medical devices have been developed for medical use, for example, intravascular use. Some of these devices include guidewires, balloon catheters, sensors and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device system for treating a heart, includes a control system including a processor and a pump, a hub coupled to the control system, a control system including a processor and a pump, a hub coupled to the control system, a first catheter shaft having a first lumen and a first end coupled to the hub, a second catheter shaft extending within the first lumen and having a first end couple to the hub, a first expandable member disposed on the first catheter shaft, wherein the first expandable member is configured to be positioned in the superior vena cava and a second expandable member disposed on the second catheter shaft distal to the first expandable member, wherein the second expandable member is configured to translate relative to the first expandable member.

Alternatively or additionally to any of the embodiments above, wherein the second expandable member is configured to be positioned in the inferior vena cava.

Alternatively or additionally to any of the embodiments above, wherein the second expandable member is configured to be positioned adjacent to a renal artery.

Alternatively or additionally to any of the embodiments above, wherein the second expandable member is configured to translate relative to the first expandable member while the first expandable member is maintained in a fixed position within the superior vena cava.

Alternatively or additionally to any of the embodiments above, wherein the second expandable member is spaced apart from the first expandable member by a variable distance, and wherein the variable distances is 12 inches to 18 inches.

Alternatively or additionally to any of the embodiments above, wherein the first lumen is configured to permit an auxiliary medical device to pass through the first lumen into the right atrium of the heart.

Alternatively or additionally to any of the embodiments above, wherein the first lumen is configured to permit an auxiliary medical device to pass from the first lumen into the right atrium of the heart while the second expandable member is positioned in the inferior vena cava.

Alternatively or additionally to any of the embodiments above, wherein the pump is designed to expand or contract the first expandable member, the second expandable member or both the first and the second expandable members based on a change in a first parameter sensed by the auxiliary medical device.

Alternatively or additionally to any of the embodiments above, wherein the first parameter is selected from the group consisting of a cardiac output of the heart, a blood flow in the pulmonary artery of the heart and a blood pressure of the pulmonary artery of the heart.

Alternatively or additionally to any of the embodiments above, wherein the auxiliary medical device includes a thermodilution catheter.

Alternatively or additionally to any of the embodiments above, further comprising a first sensing member having a first end positioned adjacent the first expandable member and a second end coupled to the control system, wherein the first sensing member is designed to sense a second parameter, and wherein the pump is designed to expand or contract the first expandable member based on a change in the first parameter, the second parameter, or a change in both the first and the second parameters.

Alternatively or additionally to any of the embodiments above, further comprising a first sensing member having a first end positioned adjacent the first expandable member and a second end coupled to the control system, wherein the first sensing member is designed to sense a second parameter, and wherein the pump is designed to expand or contract the second expandable member based on a change in the first parameter, the second parameter, or a change in both the first and the second parameters.

Alternatively or additionally to any of the embodiments above, further comprising a second sensing member having a first end positioned adjacent the second expandable member and a second end coupled to the control system, wherein the second sensing member is designed to sense a third parameter, and wherein the pump is designed to expand or contract the second expandable member based on a change in the first parameter, the second parameter, the third parameter or a change in both the first and the second parameters, a change in both the first and the third parameters, or a change in both the second and the third parameters.

Alternatively or additionally to any of the embodiments above, further comprising a second sensing member having a first end positioned adjacent the second expandable member and a second end coupled to the control system, wherein the second sensing member is designed to sense a third parameter, and wherein the pump is designed to expand or contract the first expandable member based on a change in the first parameter, the second parameter, the third parameter or a change in both the first and the second parameters, a change in both the first and the third parameters, or a change in both the second and the third parameters.

Alternatively or additionally to any of the embodiments above, wherein the second parameter, the third parameter or both the second parameter and the third parameter is a blood pressure.

Alternatively or additionally to any of the embodiments above, wherein the first sensing member, the second sensing member or both the first sensing member and the second sensing member includes a pressure wire.

Alternatively or additionally to any of the embodiments above, wherein the first sensing member, the second sensing member or both the first sensing member and the second sensing member includes a fluid-filled sensing catheter.

Another example medical device system for treating a heart includes a control system including a processor and a pump, a hub coupled to the control system, a first catheter shaft having a first lumen and a first end coupled to the hub, a second catheter shaft extending within the first lumen and having a first end coupled to the hub, wherein the second catheter shaft is configured to telescope relative to the first catheter shaft, a first expandable member disposed on the first catheter shaft, wherein the first expandable member is configured to be positioned in the superior vena cava and a second expandable member disposed on the second catheter shaft distal to the first expandable member, wherein the second expandable member is configured to be positioned in the inferior vena cava while the first expandable member is held in a fixed position within the superior vena cava.

Alternatively or additionally to any of the embodiments above, wherein the first lumen is configured to permit an auxiliary medical device to pass through the first lumen into the right atrium of the heart.

An example method for treating the heart includes advancing a medical device into the superior vena cava of the heart. Further, the medical device includes a first catheter shaft having a first lumen, a second catheter shaft extending within the first lumen, wherein the second catheter shaft is configured to translate relative to the first catheter shaft, a first expandable member disposed on the first catheter shaft, a second expandable member disposed on the second catheter shaft distal to the first expandable member, a first sensing member having a first end positioned adjacent the first expandable member, the first sensing member designed to sense a first parameter, a second sensing member having a first end positioned adjacent the second expandable member, the second sensing member designed to sense a second parameter. The method further includes positioning the first expandable member within the superior vena cava, translating the second catheter shaft toward the inferior vena cava, positioning the second expandable member within the inferior vena cava, sensing a first parameter with the first sensing member, sensing a second parameter with the second sensing member and expanding the first expandable member, the second expandable member or both the first and the second expandable members based on a change in the first parameter, a change in the second parameter, or a change in both the first and the second parameters.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example medical device system positioned in a patient;

FIG. 2 illustrates an example medical device;

FIG. 3 is a cross-sectional view taken along line 3-3 of FIG. 2;

FIG. 4 illustrates a portion of the example medical device of FIG. 2;

FIG. 5 illustrates a portion of the example medical device of FIG. 2;

FIG. 6 illustrates a portion of the example medical device of FIG. 2;

FIG. 7 is a schematic representation of the medical device of FIG. 2 positioned in the superior vena cava and the inferior vena cava;

FIG. 8 illustrates an example medical device;

FIG. 9 is an example cross-sectional view taken along line 10-10 of FIG. 8;

FIG. 10 is an example cross-sectional view taken along line 10-10 of FIG. 8;

FIG. 11 illustrates a portion of the example medical device of FIG. 8;

FIG. 12 illustrates a portion of the example medical device of FIG. 8;

FIG. 13 illustrates a portion of the example medical device of FIG. 8;

FIG. 14 is a schematic representation of the medical device of FIG. 8 positioned in the superior vena cava and the inferior vena cava;

FIG. 15 is a schematic representation of another example medical device positioned in the superior vena cava and the inferior vena cava;

FIG. 16 is a cross-sectional view taken along line 16-16 of FIG. 15;

FIG. 17 illustrates a portion of the example medical device of FIG. 15;

FIG. 18 illustrates a portion of the example medical device of FIG. 15;

FIG. 19 is a schematic representation of the medical device of FIG. 15 positioned in the superior vena cava and the inferior vena cava;

FIG. 20 illustrates an example display of the medical device system shown in FIG. 1;

FIG. 21 illustrates an example display of the medical device system shown in FIG. 1.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/of” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

The heart is an essential organ in humans, responsible for pumping blood throughout the human body. Consequently, it is fundamentally important that the mechanical pumping properties of the heart operate correctly. When the heart does not operate correctly, a variety of adverse medical conditions may arise. These adverse medical conditions may include a severe condition called acute decompensated heart failure (“ADHF”). This form of heart failure may be characterized by the sudden inability of the heart to pump efficiently. However, this inability of the heart to pump efficiently is not due to cardiac arrest, as the heart does not stop even though the heart's pumping action significantly deteriorates.

ADHF results in an inability of a failing heart to pump blood in a forward direction. Further, the inability of the heart to pump blood in a forward direction may result in excess blood backing up (e.g., collecting) in one or more chambers of the heart. For example, if a patient's left ventricle is compromised and cannot effectively pump blood into the aorta, a progressive backup of blood may eventually manifest in the right atrium. The backup of blood in the right atrium may result in a variety of adverse complications. For example, the excess blood in the right atrium may cause increased blood pressure in the right atrium, eventually resulting in the shifting of the intraventricular septum, and thereby reducing left ventricle capacitance and stroke volume. Further, conventional treatment for patients with ADHF is to treat them with intravenous diuretics until enough fluid is removed to restore hemodynamic stability. However, many patients develop diuretic resistance. Therefore, inotrope treatment and transplant is the typical treatment progression if diuretics fail.

Therefore, in some instances, it may be desirable to position and expand an expandable medical device within the superior vena cava and/or the inferior vena cava to modulate the blood flow into the right atrium, thereby allowing the excess blood in the right atrium time to vacate and, consequently, lower the blood pressure in the right atrium. Further, in addition to unloading the right side of the heart, expanding an expandable medical device within the superior vena cava and/or the inferior vena cava may also reduce renal overload, thereby increasing renal blood flow, urine output and sodium excretion, all of which may improve a patient's response to treatment with diuretics. Example medical devices designed to be positioned within the superior vena cava and/or the inferior vena cava to modulate the blood flow into the right atrium are disclosed.

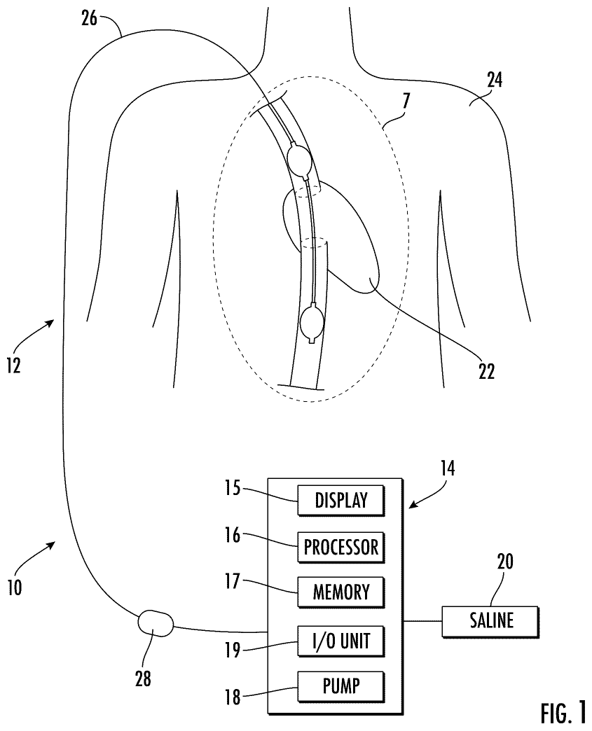

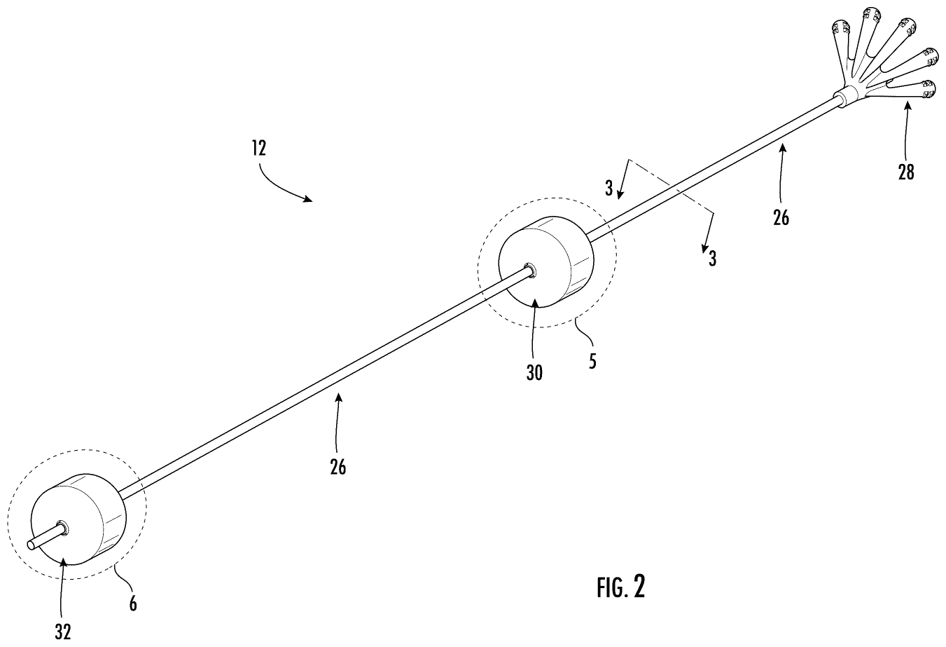

FIG. 1 illustrates an example medical device system 10. The medical device system 10 may include a medical device 12. As will be described in greater detail below with respect to FIG. 2, the medical device 12 may include a distal end region positioned proximate the heart 22 of a patient 24. The medical device 26 may include an elongate member 26 extending from a proximal end region to the distal end region of the medical device 12. Additionally, the proximal end region of the medical device 12 may include a hub 28 (e.g., manifold) coupled to the proximal end of an elongate member (e.g., catheter shaft) 26. Further, the hub 28 may be coupled to a control system 14 (e.g., console). It can be appreciated that the hub 28 shown in FIG. 1 may represent a variety of different hub configurations, some of which will be discussed herein.

The control system 14 described above may include a display 15. While FIG. 1 illustrates that the display 15 may be integrated into the control system 14, it is contemplated that the display 15 may be a separate, distinct component of the medical device system 10. In other words, the display 15 may be a separate stand-alone display, apart from the control system 14.

FIG. 1 further illustrates that the control system 14 may include, among other suitable components, one or more processors 16, a memory 17, an I/O unit 19 and a pump 18. The processor 16 of the control system 14 may include a single processor or more than one processor working individually or with one another. The processor 16 may be configured to execute instructions, including instructions that may be loaded into the memory 17 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 16 of the control system 14 may be configured to execute program instructions. Program instructions may include, for example, firmware, microcode or application code that is executed by the processor 16, a microprocessor and/or microcontroller. The one or more processors 16 may be configured to each manage different functions. They may also be configured to concurrently perform the same functions (e.g., redundant system). Further yet, they may be configured such that a processor 16 performs a given function and second processor 16 checks the result of the function of the processor 16 for correctness (e.g., command-monitor system).

The memory 17 of the control system 14 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory 17 may be or may include a non-transitory computer readable medium.

The I/O units 19 of the control system 14 may include a single I/O component or more than one I/O component each working individually or with one another. Example I/O units 40 may be any type of communication port configured to communicate with other components of the circulatory system 10. Example types of I/O units 19 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

FIG. 1 further illustrates that the control system may further include a pump 18. It can be appreciated that, in some examples, the processor 16 and the pump 18 may be located in a single control system (e.g., console, housing). However, it can be further appreciated that, in other examples, the processor 16 and the pump 18 may be separate components spaced away from one another. In either arrangement, it can be appreciated that the processor 16 and the pump 18 may be able to communicate with one another. For example, the processor 16 may communicate with the pump 18 in response to physiological changes in the patient's body. It can be further appreciated that processor 16 and the pump 18 may communicate through a variety of channels. For example, the processor 16 and the pump 18 may be hardwired with one another, or they may be wirelessly connected, or include both hardwired and wireless connections.

Additionally, in some examples the medical device system 10 may include a saline reservoir 20 (e.g., a saline bag) coupled to the control system 14. Specifically, in some examples, the saline reservoir 20 may be directly attached to the pump 18. The pump 18 may draw saline from the saline reservoir 20 in response to the processor 16 sensing physiological changes in a patient's body, as described above.

FIG. 2 illustrates the medical device 12 shown in FIG. 1. As shown in FIG. 2, the medical device 12 may include a first expandable member 30 and a second expandable member 32 disposed on the elongate member 26. In some examples, the distal end of the first expandable member 30 may be spaced from the proximal end of the second expandable member 32 about 2 inches (2″) to about 25″, or about 8″ to about 22″, or about 12″ to about 18″ or about 15″. The second expandable member 32 may be positioned distal to the first expandable member 30. In some instances, each of the first expandable member 30 and the second expandable member 32 may be referred to as an expandable medical balloon. The first expandable member 30 may include a distal end and a proximal end. Both the distal and proximal end of the first expandable member 30 may be coupled to the elongate member 26. Further, the second expandable member 32 may include a distal end and a proximal end. Both the distal and proximal end of the second expandable member 32 may be coupled to the elongate member 26.

FIG. 2 further illustrates that the proximal end of the elongate member 26 may be coupled to the hub 28. As will be described in greater detail herein, the hub 28 may include one or more ports which may be in fluid communication with the control system 14 and/or the saline reservoir 20. Additionally, as will be described in greater detail herein, the hub 28 may include one or more ports which may permit one or more medical devices (e.g., auxiliary medical devices) to be inserted therein.

It can be further appreciated that, in some examples, a portion of the elongate member 26 may pass through the expandable member 32 and extend distally beyond the expandable member 32 to form a distal tip region. However, in other examples, the medical device 12 may include a separate tip member which may be attached (e.g., bonded) to the distal waist of the expandable member 32 and/or also attached (e.g., bonded) to a portion of the elongate member 26. In other words, in some examples, the elongate member 26 itself may form the distal tip of the medical device 12. However, in other examples, a separate tip member may be attached to the expandable member 32, the elongate member 26 or both the expandable member 32 and the elongate member 26 to form the distal tip of the medical device 12.

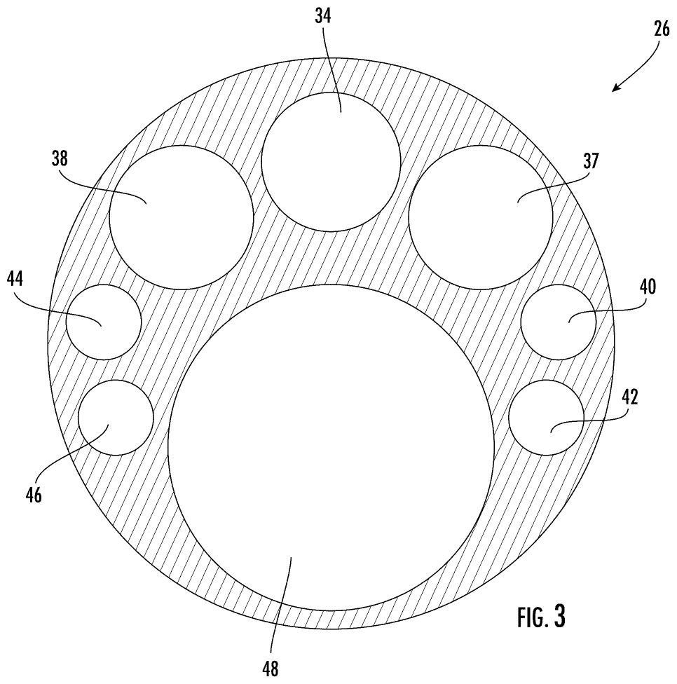

FIG. 3 illustrates a cross-section of the elongate member 26 taken along line 3-3 of FIG. 1. FIG. illustrates that the elongate member 26 may include one or more individual lumens extending therein. For example, the elongate member 26 may include a guidewire lumen 34. The guidewire lumen 34 may extend from the proximal end of the elongate member 26 to the distal end of the elongate member 26. In some examples, the guidewire lumen 34 may have a diameter of about 0.020″ to about 0.050″, or about 0.025″ to about 0.045″, or about 0.034″ to about 0.042″, or about 0.036″ to about 0.040″, or about 0.038″.

It can be appreciated that, during a medical procedure, the distal end of the elongate member 26 may be tracked over a guidewire, whereby the guidewire may pass through the guidewire lumen 34 and exit the medical device 12 through a port of the hub 28. In other words, the guidewire lumen 34 may permit the medical device 12 to be tracked over a guidewire (previously positioned within a patient) to a treatment site (e.g., the heart) within the patient.

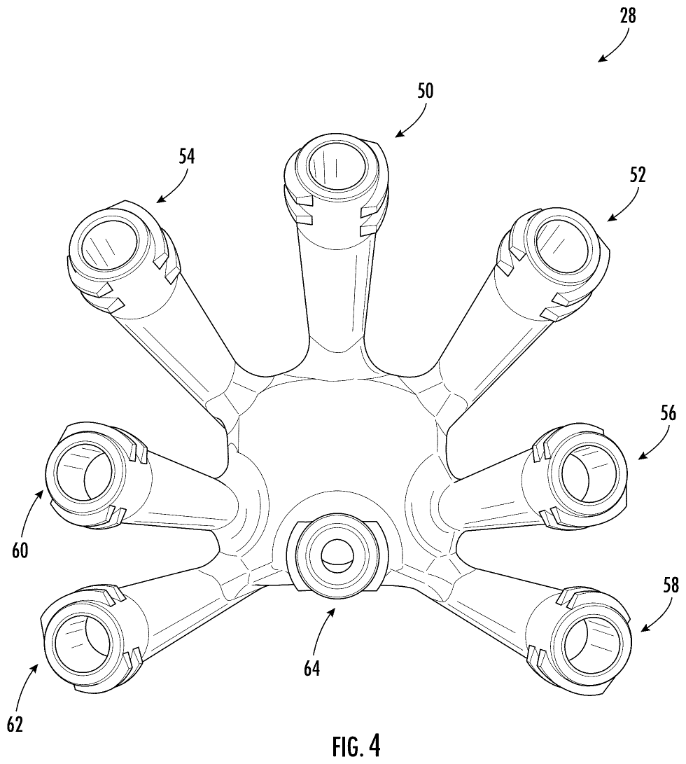

FIG. 3 further illustrates that the elongate member 26 may include a first inflation lumen 37 and a second inflation lumen 38, each of which may be in fluid communication with the pump 18 (which, in turn, may be in fluid communication with the saline reservoir 20). In some examples, the first inflation lumen 37 may be in fluid communication with the first expandable member 30. It can be appreciated that the saline from the saline reservoir 20 may be passed through a first inflation port (shown in FIG. 4) on the hub 28, through the first inflation lumen 37 and into the first expandable member 30 to expand or deflate the first expandable member 30. In some examples, the first inflation lumen 37 may have a diameter of about 0.020″ to about 0.055″, or about 0.025″ to about 0.050″, or about 0.030″ to about 0.046″, or about 0.036″ to about 0.042″, or about 0.040″. Similarly, in some examples, the second inflation lumen 38 may be in fluid communication with the second expandable member 32. It can be appreciated that the saline from the saline reservoir 20 may be passed through a second inflation port (shown in FIG. 4) on the hub 28, through the second inflation lumen 38 and into the second expandable member 32 to expand or deflate the second expandable member 32. In some examples, the second inflation lumen 38 may have a diameter of about 0.020″ to about 0.055″, or about 0.025″ to about 0.050″, or about 0.030″ to about 0.046″, or about 0.036″ to about 0.042″, or about 0.040″.

FIG. 3 further illustrates that the elongate member 26 may include one or more additional lumens 40, 42, 44, 46 which are configured to permit a sensing member to extend therein. For example, FIG. 3 illustrates a first sensing member lumen 40, which may be designed to permit a sensing member to extend through the elongate member 26 to a position whereby the sensing member is proximal to the first expandable balloon 30 and within the superior vena cava. FIG. 3 further illustrates a second sensing member lumen 42, which may be designed to permit a sensing member to extend through the elongate member 26 to a position whereby the sensing member is distal to the first expandable balloon 30 and within the superior vena cava. FIG. 3 further illustrates a third sensing member lumen 44, which may be designed to permit a sensing member to extend through the elongate member 26 to a position whereby the sensing member is proximal to the second expandable balloon 32 and within the inferior vena cava. FIG. 3 further illustrates a fourth sensing member lumen 46, which may be designed to permit a sensing member to extend through the elongate member 26 to a position whereby the sensing member is distal to the second expandable balloon 30 and within the inferior vena cava. In some examples, the sensing member lumens 40, 42, 44, 46 may each have a diameter of about 0.005″ to about 0.040″, or about 0.010″ to about 0.030″, or about 0.015″ to about 0.025″, or about 0.018″ to about 0.023″, or about 0.021″.

FIG. 3 further illustrates that the elongate member 26 may include a working channel 48 (e.g., working lumen). In some examples, the working channel 48 may extend from the proximal end of the elongate member 26 to the distal end of the elongate member 26. In other examples, the working channel 48 may extend from the proximal end of the elongate member 26 to a position proximal of the distal end of the elongate member 26. For example, the working channel 48 may extend from the proximal end of the elongate member 26 to a position adjacent the distal end of the first expandable member 30. As will be described in greater detail below, the medical device 12 may be designed to permit an auxiliary medical device (e.g., diagnostic medical device, thermodilution catheter, etc.) to pass through the hub 28 and into the working channel 48, whereby the auxiliary device may pass through an aperture in the wall of the elongate member 26 and into the right atrium and/or the pulmonary artery of a patient. In some examples, the working channel 48 may have a diameter of about 0.075″ to about 0.110″, or about 0.080″ to about 0.105″, or about 0.085″ to about 0.095″, or about 0.088″ to about 0.092″, or about 0.090″.

In some examples, the wall thickness between adjacent lumens 34, 37, 38, 40, 42, 44, 46, 48 of the elongate member 26 illustrated in FIG. 3 may be about 0.0015″ to about 0.0085″, or about 0.0025″ to about 0.0075″, or about 0.0035″ to about 0.0065″, or about 0.0045″ to about 0.0055″, or about 0.0050″.

FIG. 4 illustrates the example hub 48 (e.g., manifold) of the medical device 12. FIG. 4 illustrates that the hub 28 may include eight separate access ports 50, 52, 54, 56, 58, 60, 62, 64 (e.g. apertures, openings, etc.). While FIG. 4 illustrates the hub 28 including eight access ports 50, 52, 54, 56, 58, 60, 62, 64 it can be appreciated that the hub 28 may include 1, 2, 3, 4, 5, 6, 7, 8, 9, 10 or more access ports.

FIG. 4 further illustrates that the hub 48 may include a guidewire port 50. The guidewire port 50 may be in fluid communication with guidewire lumen 34 described above. The guidewire port 50 may permit a guidewire to be inserted therethrough and into the guidewire lumen 34.

FIG. 4 further illustrates that the hub 48 may include a first inflation port 52. The first inflation port 52 may be in fluid communication with the first inflation lumen 37 described above. The first inflation port 52 may permit the passing of saline (or other inflation media) through the elongate member 26 and into the first expandable member 30. FIG. 4 further illustrates that the hub 48 may include a second inflation port 54. The second inflation port 54 may be in fluid communication with the second inflation lumen 38 described above. The second inflation port 54 may permit the passing of saline (or other inflation media) through the elongate member 26 and into the second expandable member 32.

FIG. 4 further illustrates that the hub 48 may include one or more sensing member ports 56, 58, 60, 62. For example, FIG. 4 further illustrates that the hub 28 may include a first sensing member port 56, which may be in fluid communication with the sensing member lumen 40. FIG. 4 further illustrates that the hub 28 may include a second sensing member port 58, which may be in fluid communication with the sensing member lumen 42. FIG. 4 further illustrates that the hub 28 may include a third sensing member port 60, which may be in fluid communication with the sensing member lumen 44. FIG. 4 further illustrates that the hub 28 may include a fourth sensing member port 62, which may be in fluid communication with the sensing member lumen 46.

FIG. 4 further illustrates that the hub 28 may include a working channel port 64, which may be in fluid communication with the working channel 48. The working channel port 64 may permit an auxiliary medical device to pass through the hub 28 and into the working channel 48, whereby the auxiliary medical device may eventually pass through an aperture in the wall of the elongate member 26 and into the right atrium and/or the pulmonary artery of a patient.

It can be appreciated that each of the separate access ports 50, 52, 54, 56, 58, 60, 62, 64 on the hub 28 may include a threaded region which may permit a user to attach medical instruments thereto. For example, the threaded region on each of the first inflation port 52 and the second inflation port 54 may permit a user to attach the hub 28 to the control system 14, the pump 18 and/or the saline reservoir 20, whereby the pump 18 and the saline reservoir may be utilized to inflate or deflate the first expandable member 30 and/or the second expandable member 32.

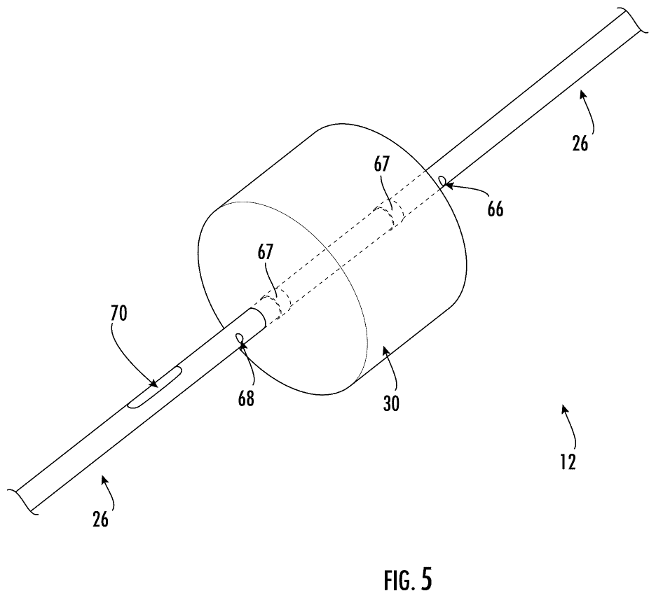

FIG. 5 illustrates a detailed view of a portion of the medical device 12 shown in FIG. 2. For example, FIG. 5 illustrates the first expandable member 30 attached to the elongate member 26. FIG. 5 illustrates that the medical device 26 may include one or more apertures positioned along the elongate member 26. In some instances, the one or more apertures positioned along the elongate member may be positioned proximate to the first expandable member 30. For example, FIG. 5 illustrates that the medical device 26 may include a sensing member aperture 66 positioned proximal to the first expandable member 30. The aperture 66 may extend through the wall of the elongate member 26. Further, it can be appreciated that the aperture 66 may be in fluid communication with the sensing member lumen 40 described herein. FIG. 5 further illustrates that the medical device 26 may include a sensing member aperture 68 positioned distal to the first expandable member 30. The aperture 68 may extend through the wall of the elongate member 26. Further, it can be appreciated that the aperture 68 may be in fluid communication with the sensing member lumen 42 described herein. FIG. 5 further illustrates that the medical device 26 may include one or more marker bands 67 positioned underneath the first expandable member 30 to aid in placement of the first expandable member 30 at the target treatment site.

FIG. 5 further illustrates that the medical device 26 may include a working channel aperture 70 positioned distal to the first expandable member 30. In some examples, the proximal end of the aperture 70 may be spaced away from the distal end of the first expandable member 30 about 0.25″ to about 2.25″, or about 0.50″ to about 1.75″, or about 0.75″ to about 1.5″, or about 0.85″ to about 1.25″ or about 1.0″. The aperture 70 may extend through the wall of the elongate member 26. Further, it can be appreciated that the aperture 70 may be in fluid communication with the working channel 48 described herein. Accordingly, it can be further appreciated that the working channel aperture 70 may permit an auxiliary medical device which has been inserted into the hub 28 and passed through the working channel 48 to pass through the wall of the elongate member 26, whereby the auxiliary medical device may extend away from the medical device 26 to access other portions of a patient's anatomy. In some instances, a thermodilution catheter (e.g., Swan-Ganz catheter) may be inserted into the hub 28, passed through the working channel 48, through the working channel aperture 70, whereby the thermodilution catheter may extend away from the medical device 26 and access the right atrium and/or the pulmonary artery of a patient. It is contemplated that other auxiliary medical devices (in addition to a thermodilution catheter) may be passed through the working channel 48 to access portions of a patient's anatomy.

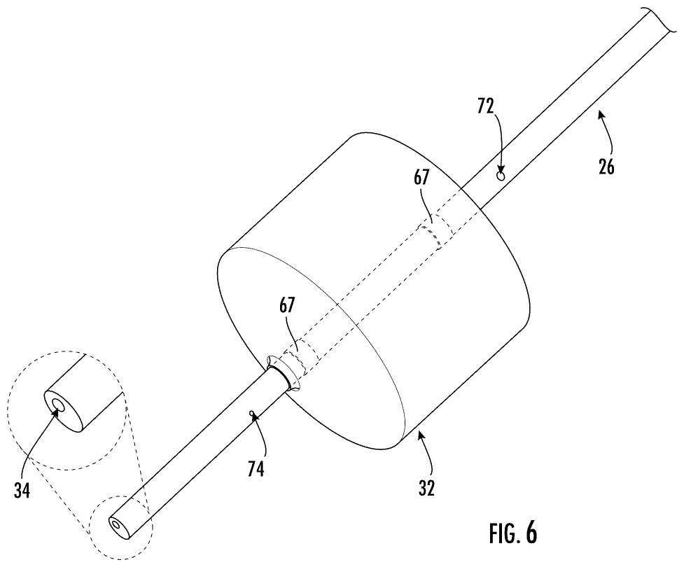

FIG. 6 illustrates a detailed view of a portion of the medical device 12 shown in FIG. 2. For example, FIG. 6 illustrates the second expandable member 32 disposed on the elongate member 26. FIG. 6 illustrates that the medical device 26 may include one or more apertures positioned along the elongate member 26. In some instances, the one or more apertures positioned along the elongate member 26 may be positioned proximate to the second expandable member 32. For example, FIG. 6 illustrates that the medical device 26 may include a sensing member aperture 72 positioned proximal to the first expandable member 32. The aperture 72 may extend through the wall of the elongate member 26. Further, it can be appreciated that the aperture 72 may be in fluid communication with the sensing member lumen 44 described herein. FIG. 5 further illustrates that the medical device 26 may include a sensing member aperture 74 positioned distal to the second expandable member 32. The aperture 74 may extend through the wall of the elongate member 26. Further, it can be appreciated that the aperture 74 may be in fluid communication with the sensing member lumen 44 described herein.

It is contemplated that the shape of the sensing member apertures 66, 68, 70, 72 may be square, triangular, rectangular, ovular, polygonal, combinations thereof or any other suitable geometric shape.

Further, as described herein, in some examples each of the sensing member apertures 66, 68, 72, 74 described herein may each extend through the wall of the elongate member 26. However, in other examples it is contemplated that the medical device 12 may include a membrane which extends along the outer surface of the elongate member 26, whereby the membrane extends over each of the sensing member apertures 66, 68, 72, 74. In these examples, it can be appreciated that a membrane covering any of the sensing member apertures 66, 68, 70, 72 may be substantially flush with the outer surface of the elongate member 26. Further, the membrane extending across any of the sensing member apertures 66, 68, 72, 74 may permit the sensing member lumens 40, 42, 44, 46 in fluid communication with the sensing member apertures 66, 68, 72, 74 to be filled with fluid (e.g., the membrane may maintain the fluid within a fluid column defined by the sensing member lumens), whereby each of the fluid filled lumens may communicate with a pressure sensor of the medical device system 12. It can be appreciated that a change in force occurring along the membrane of any of the sensing member apertures 66, 68, 72, 74 may be transmitted through its respective fluid filled sensing member lumen 40, 42, 44, 46 to a pressure sensor positioned in the control system 14. The pressure sensor may then send a signal to the processor 16 of the control system in response to a change in pressure occurring at the membrane of any of the sensing member apertures 66, 68, 72, 74.

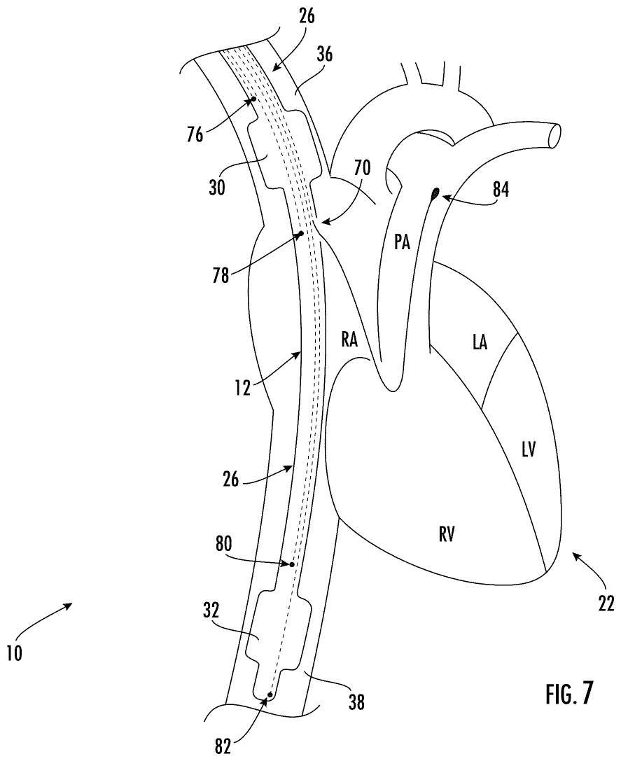

FIG. 7 illustrates the detailed view of FIG. 1. In particular, FIG. 7 illustrates the first expandable member 30 positioned in the superior vena cava 36 and the second expandable member 32 positioned in the inferior vena cava. While FIG. 7 illustrates that second expandable member 32 positioned in the inferior vena cava, in other examples it is contemplated that the second expandable member 32 may be positioned proximate the renal arteries or renal veins. Further, FIG. 7 illustrates an auxiliary medical device 84 (e.g., diagnostic catheter, thermodilution catheter, auxiliary sensing member, etc.) extending within the working channel 48, passing through the working channel aperture 70, extending through the right atrium (RA) and into the pulmonary artery (PA) of the patient.

As discussed herein, the elongate member 26 may include one or more individual lumens extending therein. For example, it can be appreciated that the elongate member 26 may include a first inflation lumen 37 (shown in FIG. 3) which may extend from the first expandable member 30 and be in fluid communication with the pump 18, which, in turn, may be in fluid communication with the saline reservoir 20. Additionally, as discussed herein, the elongate member 26 may include a second inflation lumen 38 (shown in FIG. 3) which may extend from the second expandable member 32 and be in fluid communication with the pump 18, which, in turn, may be in fluid communication with the saline reservoir 20.

Additionally, FIG. 7 illustrates that the elongate member 26 may include one or more additional lumens (in addition to the inflation lumens 37, 38 described above), each of which may be configured to permit a sensing member to extend therein. For example, FIG. 7 illustrates a sensing member 76 extending within the sensing member lumen 40, whereby the distal end of the sensing member 76 may be positioned proximal of the first expandable member 30. FIG. 7 further illustrates a sensing member 78 extending within the sensing member lumen 42, whereby the distal end of the sensing member 78 may be positioned distal of the first expandable member 30. FIG. 7 further illustrates a sensing member 80 extending within the sensing member lumen 44, whereby the distal end of the sensing member 80 may be positioned proximal of the second expandable member 32. FIG. 7 further illustrates a sensing member 82 extending within the sensing member lumen 46, whereby the distal end of the sensing member 82 may be positioned distal of the second expandable member 32.

FIG. 7 illustrates the sensing members 76, 78 positioned proximate the first expandable member 30 in the superior vena cava 36. FIG. 7 further illustrates the sensing members 80, 82 positioned proximate the second expandable member 32 in the inferior vena cava 38. It can be appreciated from FIG. 7 that, when positioned proximate a target treatment site, the distal end of the sensing members 76, 78 may align with the sensing member apertures 66, 68, respectively. Similarly, it can be appreciated from FIG. 7 that, when positioned proximate a target treatment site, the distal end of the sensing members 80, 82 may align with the sensing member apertures 70, 72, respectively. In some examples, all of the sensing members 76, 78, 80, 82 may be configured to sense a change in one or more physiological parameters/characteristics occurring in a patient 24. Specifically, in some examples, the sensing members 76, 78, 80, 82 may include pressure sensing capabilities. In other words, the sensing members 76/78 may include a pressure sensor designed to measure the central venous pressure in the superior vena cava 36 and the sensing members 80/82 may include a pressure sensor designed to measure the inferior venous pressure in the inferior vena cava 38.

It can be appreciated that any of the sensing members 76, 78, 80, 82 may include a variety of different configurations. For example, in some instances, the sensing members 76, 78, 80, 82 may include a pressure sensing wire which is integrated into the control system 14. In other examples, the sensing members 76, 78, 80, 82 may be an invasive blood pressure sensor (IBP sensor). Additionally, in some examples, each of the sensing members may include a microelectricalmechanical (MEMS) sensor coupled to an elongated wire. The MEMS pressure sensor may be able to detect and respond rapidly to very small changes in blood pressure. Further, a MEMS pressure sensor may be able to transmit a signal to the processor 16 indicating there has been a change in blood pressure in the region in which the MEMS sensor is disposed (e.g., the superior vena cava, inferior vena cava, proximate the right atrium, etc.).

In other examples, each of the sensing members 76, 78, 80, 82 may include a pressure sensing catheter, whereby the catheter includes a fluid-filled lumen, the distal end of which may be open to the surrounding environment. Further, a change in pressure in the area surrounding the distal end of the fluid-filled catheter may cause the fluid within the catheter to shift. This shifting of the fluid within the fluid-filled catheter may be sensed by the processor 16.

Additionally, in yet other examples, each of the sensing members 76, 78, 80, 82 may include a fiber optic pressure sensing catheter, whereby the fiber optic pressure sensing catheter includes a fiber optic pressure sensor. The fiber optic pressure sensor may sense a change in blood pressure (in areas adjacent to the sensor) based on a change in the light intensity surrounding the sensor. This change in light intensity may be sensed by the processor 16. In any of the examples discussed herein, the sensing members 76, 78, 80, 82 may include a variety of sensors. For example, in addition to the sensors discussed above, the sensing members 76, 78, 80, 82 disclosed herein may include piezo-resistive sensors, piezo-capacitive sensors, pressure sensors, flow sensors, accelerometers, temperature sensors, or the like.

As discussed herein, if the pumping action of the heart 22 is compromised due to a weakened left ventricle (for example), blood may begin to back up therein. Further, blood backing up in the left ventricle may result in blood backing up in the left atrium. Further yet, blood backing up in the left atrium may result in blood backing up within the pulmonary veins, the lungs and the pulmonary arteries. Blood backing up in the pulmonary arteries may further result in blood backing up the right ventricle which, over time, results in the backing up of blood (and increased blood pressure) in the right atrium and areas immediately adjacent to the right atrium (e.g., the superior vena cava, the inferior vena cava).

Further, it can be appreciated that as the blood backs up into the right atrium, additional blood may continue to flow into the right atrium from the superior vena cava and the inferior vena cava. Therefore, to mediate the adverse effects caused by the backing up of blood into the right atrium (e.g., the increase in blood volume and blood pressure within the right atrium), it may be desirable to temporarily occlude (fully or partially) the superior vena cava and/or inferior vena cava.

Therefore, in some examples, one or more of the sensing members 76, 78, 80, 82 and/or auxiliary medical device 84 may sense a change in a physiological parameter (e.g., a change in blood pressure, cardiac output, stroke volume, blood flow, HR, MAP, etc.) in the areas surrounding the superior vena cava 36, the inferior vena cava 38, the right atrium and/or pulmonary artery. Further, it can be appreciated that any sensor sensing a change in the parameter may transmit a signal to the processor 16. The processor 16 may include a memory and/or an algorithm which is designed to measure, interpret, process, analyze, compute, evaluate, etc. the signal received from the one or more sensing members 76, 78, 80, 82 and/or auxiliary medical device 84. Further yet, the algorithm may process the signal received from the one or more sensing members 76, 78, 80, 82 and/or the auxiliary medical device 84 and, if necessary, automatically communicate with the pump 18 to fill or evacuate fluid from the first expandable member 30 and/or the second expandable member 32. As discussed above, the pump 18 may draw fluid from the saline reservoir 20 to expand the first expandable member 30 and/or the second expandable member 32.

It can be appreciated that when the sensing members 76, 78, 80, 82 and/or the auxiliary medical device 84 may sense a change in a physiological parameter (e.g., a change in blood pressure, cardiac output, stroke volume, blood flow, HR, MAP, etc.) in the areas surrounding the superior vena cava, the inferior vena cava, the right atrium and/or pulmonary artery expanding the first expandable member 30 and/or the second expandable member 32 may restrict the flow of blood into the areas surrounding the right atrium. Restricting the flow of blood into the areas surrounding the right atrium may allow excess blood which has built up in the right atrium to vacate (or partially vacate) the right atrium, thereby reducing the blood pressure built up in the heart 22.

In some instances, it may not be necessary for the system 10 to expand the first expandable member 30 and/or the second expandable member 32 to a point in which the expandable members 30, 32 completely occlude the superior vena cava 36 and the inferior vena cava 38, respectively. Rather, in some instances the processor 16 may process the signals received from one or more of the sensing members 76, 78, 80, 82 and/or the auxiliary medical device 84 and only partially occlude the superior vena cava 36 and/or the inferior vena cava 38. In other instances, the processor 16 may process the signals received from one or more of the sensing members the sensing members 76, 78, 80, 82 and/or the auxiliary medical device 84 and fully occlude the superior vena cava 36 or the inferior vena cava 38 while leaving the other of the superior vena cava 36 and the inferior vena cava 38 open or only partially occluded. It can be appreciated that the processor 16 may include an algorithm which is designed to analyze varying physiological parameters occurring in the superior vena cava, the inferior vena cava, the right atrium and/or the pulmonary artery to determine the degree to which either the first expandable member 30 and/or the second expandable member 32 should be occluded (if at all).

It is noted that the physiological parameters discussed above which may be processed by the processor 16 to assess the degree to which the first expandable member
and/or the second expandable member 32 should be occluded is not limited to merely the blood pressure in the superior vena cava 36, the inferior vena cava 38, the right atrium and/or the pulmonary artery. Rather, the processor 16 may assess blood flow, blood pressure, cardiac output, stroke volume, HR, MAP, motion of the inferior vena cava, motion of the superior vena cava, respiration cycles, volume in the inferior vena cava, volume in the superior vena cava or the like.

Further, in some instances the algorithm utilized by the processor 16 may include an assessment of the blood pressure readings in the superior vena cava 36, the inferior vena cava 38, the right atrium and/or the pulmonary artery for a particular patient, whereby the algorithm may further utilize a look-up table or artificial intelligence algorithm to determine the degree to which either the first expandable member 30 and/or the second expandable member 32 should be occluded for that particular patient at a given time point.

Further yet, in some examples the control system 14 may also be coupled to a sensor which is providing a patient's ECG to the processor 16. In that case, the cardiac cycle timing (e.g., the occlusion duty cycle of may be timed to features of the ECG) of a particular patient may be utilized to determine the degree to which either the first expandable member 30 and/or the second expandable member 32 should be occluded for that particular patient at a given time point.

As discussed above, the timing of the inflation or evacuation of the first expandable member 30 may be different than the timing of inflation or evacuation of the second expandable member 32. In other words, the inflation or evacuation of the first expandable member 30 may not be in sync with the inflation or evacuation of the second expandable member 32. Permitting the inflation or evacuation of the first expandable member 30 to be out of sync with the second expandable member 32 may allow for a minimal impact on cerebral blood flow without raising renal or hepatic venous pressures and reducing cardiac workload while maintaining overall mean arterial pressures. The inflation or evacuation of the first expandable member 30 and the second expandable member 32 may be actuated intermittently, continuously, synchronously, asynchronously, individually or automatically based on various physiological parameters (e.g., blood pressure, cardiac output, stroke volume, arterial pressure, venous pressure, superior vena cava pressure, inferior vena cava pressure, right atrial pressure, pulmonary artery pressure, mean arterial pressure, heart rate, renal artery pressure, renal vein pressure).

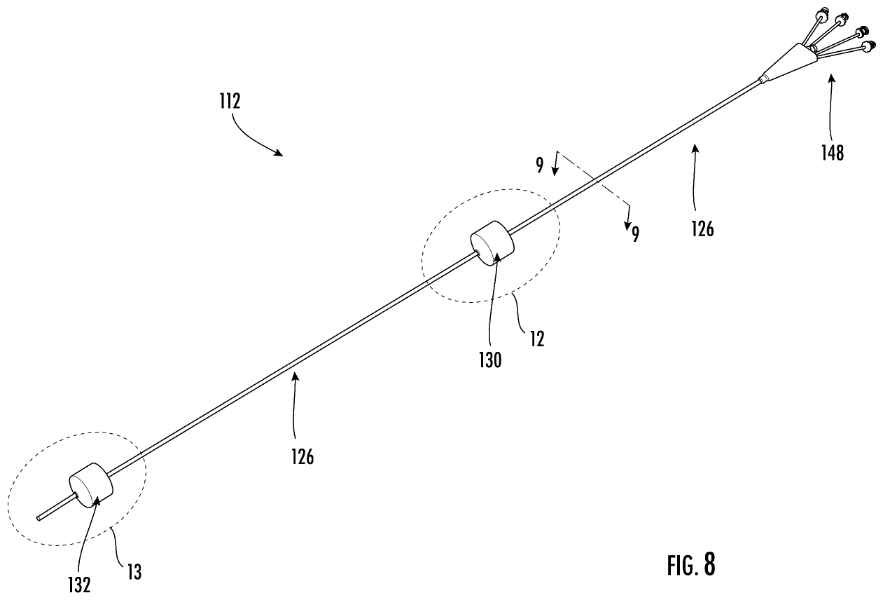

FIG. 8 illustrates another example medical device 112 which may be utilized with the system 10 described herein. The medical device 112 shown in FIG. 8 may be similar in form and function to the medical device 12 illustrated in FIG. 1. For example, the medical device 112 may include a first expandable member 130 and a second expandable member 132 disposed on the elongate member 126. In some examples, the distal end of the first expandable member 130 may be spaced from the proximal end of the second expandable member 132 about 2″ to about 25″, or about 8″ to about 22″, or about 12″ to about 18″ or about 15″. The second expandable member 132 may be positioned distal to the first expandable member 130. In some instances, each of the first expandable member 130 and the second expandable member 132 may be referred to as an expandable medical balloon. The first expandable member 130 may include a distal end and a proximal end. Both the distal and proximal end of the first expandable member 130 may be coupled to the elongate member 126. Further, the second expandable member 1-32 may include a distal end and a proximal end. Both the distal and proximal end of the second expandable member 132 may be coupled to the elongate member 126.

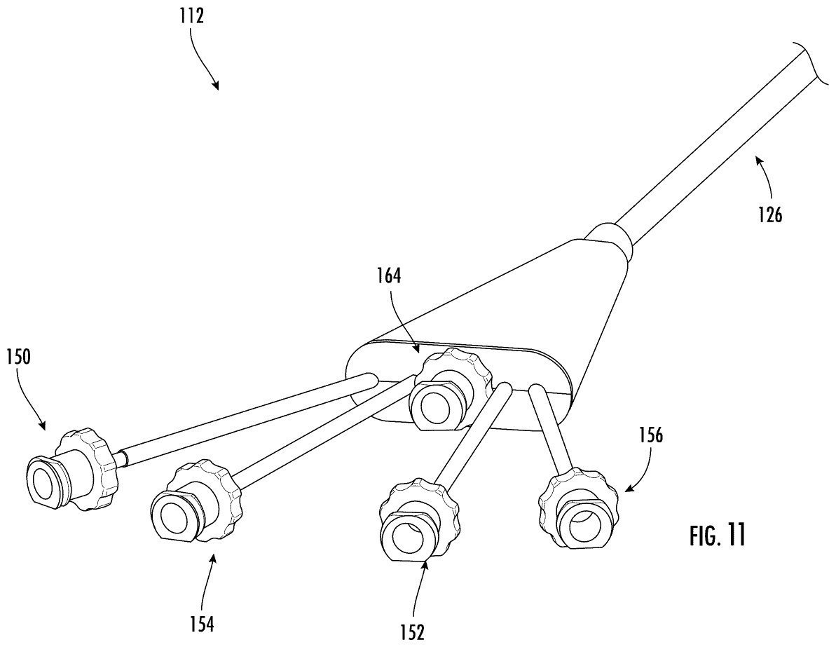

FIG. 8 further illustrates that the proximal end of the elongate member 126 may be coupled to a hub 128. As will be described in greater detail with respect to FIG. 11, the hub 128 may include one or more ports which may be in fluid communication with the control system 14 and/or the saline reservoir 20. Additionally, as will be described in greater detail herein, the hub 128 may include one or more ports which may permit one or more medical devices (e.g., auxiliary medical devices) to be inserted therein.

It can be further appreciated that while, in some examples, the elongate member 126 may pass through the expandable member 132 to form a distal tip member of the medical device 112. In other examples, the medical device 112 may include a separate tip member which is attached (e.g., bonded) to the distal waist of the expandable member 132 and/or also attached (e.g., bonded) to the elongate member 126. In other words, in some examples, the elongate member 126 itself may form the distal tip of the medical device 112, however, in other examples, a separate tip member may be attached to the expandable member 132, the elongate member 126 or both the expandable member 132 and the elongate member 126 to form the distal tip of the medical device 112.

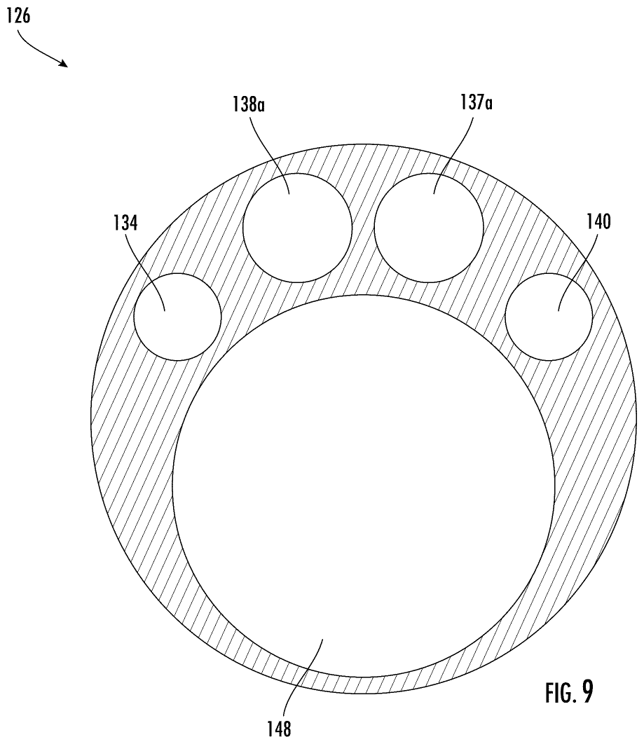

FIG. 9 illustrates a cross-section of the elongate member 26 taken along line 9-9 of FIG. 8. FIG. 9 illustrates that the elongate member 126 may include one or more individual lumens extending therein. For example, the elongate member 126 may include a combination guidewire and sensing member lumen 134. The combination guidewire and sensing member lumen 134 may extend from the proximal end of the elongate member 126 to the distal end of the elongate member 126. In some examples, the guidewire lumen 134 may have a diameter of about 0.005″ to about 0.040″, or about 0.010″ to about 0.030″, or about 0.015″ to about 0.025″, or about 0.018″ to about 0.023″, or about 0.021″.

It can be appreciated that, during a medical procedure, the distal end of the elongate member 126 may be tracked over a guidewire, whereby the guidewire may pass through the combination guidewire and sensing member lumen 134 and exit the medical device 112 through a port of the hub 128. In other words, the combination guidewire and sensing member lumen 134 may permit the medical device 112 to be tracked over a guidewire (previously positioned within a patient) to a treatment site (e.g., the heart) within the patient. As will be described in greater detail below, after the medical device 112 is tracked over a guidewire (previously positioned within a patient) to a treatment site, the guidewire may be removed from the patient, whereby the combination guidewire and sensing member lumen 134 may be utilized to pass a sensing member therethrough to a position proximate the treatment site.

FIG. 9 further illustrates that the elongate member 126 may include a first inflation lumen 137a and a second inflation lumen 138a, each of which may be in fluid communication with the pump 18 (which, in turn, may be in fluid communication with the saline reservoir 20). In some examples, the first inflation lumen 137a may be in fluid communication with the first expandable member 130. It can be appreciated that the saline from the saline reservoir 20 may be passed through a first inflation port (shown in FIG. 11) on the hub 128, through the first inflation lumen 137a and into the first expandable member 130 to expand or deflate the first expandable member 130. In some examples, the first inflation lumen 137a may have a diameter of about 0.010″ to about 0.045″, or about 0.015″ to about 0.040″, or about 0.020″ to about 0.036″, or about 0.026″ to about 0.034″, or about 0.032″. Similarly, in some examples, the second inflation lumen 138a may be in fluid communication with the second expandable member 132. It can be appreciated that the saline from the saline reservoir 20 may be passed through a second inflation port (shown in FIG. 11) on the hub 128, through the second inflation lumen 138a and into the second expandable member 132 to expand or deflate the second expandable member 132. In some examples, the second inflation lumen 138a may have a diameter of about 0.010″ to about 0.045″, or about 0.015″ to about 0.040″, or about 0.020″ to about 0.036″, or about 0.026″ to about 0.034″, or about 0.032″.

FIG. 9 further illustrates that the elongate member 26 may include a lumen 140 and the combination guidewire and sensing lumen 134 each of which are configured to permit a sensing member to extend therein. For example, FIG. 9 illustrates a sensing member lumen 140, which may be designed to permit a sensing member to extend through the elongate member 126 to a position whereby the sensing member is proximal to the first expandable balloon 130 and within the superior vena cava. FIG. 9 further illustrates the combination guidewire and sensing member lumen 134, which may be designed to permit a sensing member to extend through the elongate member 126 to a position whereby the sensing member extends out of the distal end of the elongate lumen 126 to a position distal to the second expandable balloon 132 within the inferior vena cava. In some examples, the combination guidewire lumen and the sensing member lumen 140 may each have a diameter of about 0.005″ to about 0.040″, or about 0.010″ to about 0.030″, or about 0.015″ to about 0.025″, or about 0.018″ to about 0.023″, or about 0.021″.

FIG. 9 further illustrates that the elongate member 126 may include a working channel 148 (e.g., working lumen). In some examples, the working channel 148 may extend from the proximal end of the elongate member 126 to the distal end of the elongate member 126. In other examples, the working channel 148 may extend from the proximal end of the elongate member 126 to a position proximal of the distal end of the elongate member 126. For example, the working channel 148 may extend from the proximal end of the elongate member 126 to a position adjacent the distal end of the first expandable member 130. As will be described in greater detail below, the medical device 112 may be designed to permit an auxiliary medical device (e.g., diagnostic device, thermodilution catheter, etc.) to pass through the hub 128, through the working channel 148, through an aperture in the wall of the elongate member 126 and into the right atrium and/or the pulmonary artery of a patient. In some examples, the working channel 148 may have a diameter of about 0.095″ to about 0.130″, or about 0.100″ to about 0.125″, or about 0.105″ to about 0.115″, or about 0.108″ to about 0.112″, or about 0.110″.

In some examples, the wall thickness between adjacent lumens 134, 137a, 138a, 140, 148 of the elongate member 126 illustrated in FIG. 9 may be about 0.0015″ to about 0.0085″, or about 0.0025″ to about 0.0075″, or about 0.0035″ to about 0.0065″, or about 0.0045″ to about 0.0055″, or about 0.0050″.

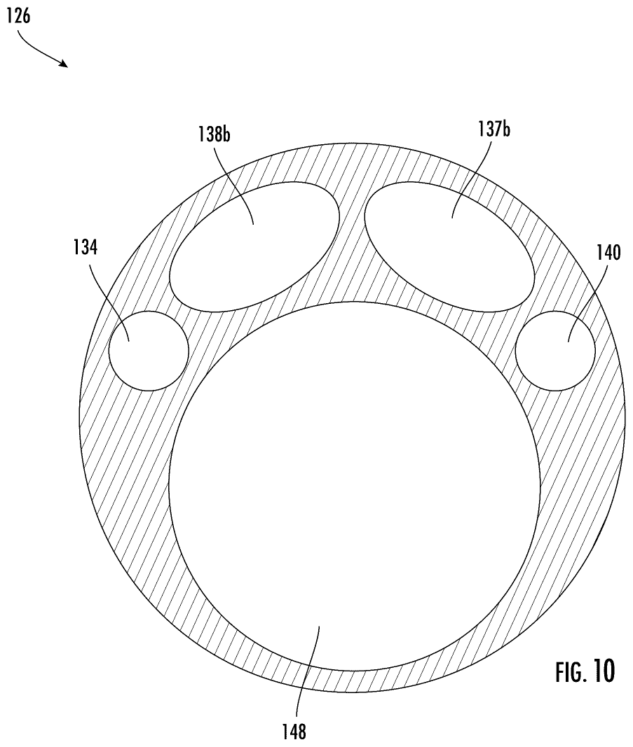

FIG. 10 illustrates another example cross-section of the elongate member 126 taken along line 9-9 of FIG. 8. FIG. 10 illustrates the elongate member 126 may include the combination guidewire and sensing lumen 134, a first inflation lumen 137b, a second inflation lumen 138b and a sensing member lumen 140. However, FIG. 10 illustrates that, in some examples, the cross-sectional shape of the first inflation lumen 137b and/or the second inflation lumen 138b may be ovular. However, this is not intended to be limiting. It is contemplated that the cross-sectional shape of the first inflation lumen 137b and/or the second inflation lumen 138b may be square, triangular, rectangular, polygonal, combinations thereof or any other suitable geometric shape.

In some examples, the wall thickness between adjacent lumens 134, 137b, 138b, 140, 148 of the elongate member 126 illustrated in FIG. 10 may be about 0.0015″ to about 0.0085″, or about 0.0025″ to about 0.0075″, or about 0.0035″ to about 0.0065″, or about 0.0045″ to about 0.0055″, or about 0.0050″.

FIG. 11 illustrates the example hub 148 (e.g., manifold) of the medical device 112. FIG. 11 illustrates that the hub 128 may include five separate access ports 150, 152, 154, 156, 164 (e.g. apertures, openings, etc.). While FIG. 11 illustrates the hub 128 including five access ports 150, 152, 154, 156, 164 it can be appreciated that the hub 128 may include 1, 2, 3, 4, 5, 6, 7, 8, 9, 10 or more access ports.

FIG. 11 further illustrates that the hub 148 may include a combination guidewire and sensing member port 150. The combination guidewire and sensing member port 150 may be in fluid communication with combination guidewire and sensing member lumen 134 described above. The combination guidewire and sensing member port 150 may permit a guidewire to be inserted therethrough and into the combination guidewire and sensing member lumen 134. As described above, after the medical device 112 has been tracked over the guidewire to the target treatment site, the guidewire may be removed from the combination guidewire and sensing member lumen 134, whereby a sensing member may be passed through the combination guidewire and sensing member port 150 and into the combination guidewire and sensing member lumen 134.

FIG. 11 further illustrates that the hub 148 may include a first inflation port 152. The first inflation port 152 may be in fluid communication with the first inflation lumen 137a, 137b described above. The first inflation port 152 may permit the passing of saline (or other inflation media) through the elongate member 126 and into the first expandable member 130. FIG. 11 further illustrates that the hub 148 may include a second inflation port 154. The second inflation port 154 may be in fluid communication with the second inflation lumen 138a, 138b described above. The second inflation port 154 may permit the passing of saline (or other inflation media) through the elongate member 126 and into the second expandable member 132.

FIG. 11 further illustrates that the hub 48 may include a sensing member port 156 and the combination guidewire and sensing port 150 which are each configured to permit a sensing member to extend therein. For example, FIG. 11 illustrates that that the sensing member port 156 may be in fluid communication with the sensing member lumen 140.

FIG. 11 further illustrates that the hub 128 may include a working channel port 164, which may be in fluid communication with the working channel 148. The working channel port 164 may permit an auxiliary medical device (e.g., diagnostic medical device, thermodilution catheter, etc.) to pass through the hub 128 and into the working channel 148, whereby the auxiliary medical device may eventually pass through an aperture in the wall of the elongate member 126 and into the right atrium and/or the pulmonary artery of a patient.

It can be appreciated that each of the separate access ports 150, 152, 154, 156, 164 of the hub 128 may include a threaded region which may permit a user to attach medical instruments thereto. For example, the threaded region on each of the first inflation port 152 and the second inflation port 154 may permit a user to attach the hub 128 to the pump 18 and/or the saline reservoir 20, whereby the pump 18 and the saline reservoir 20 may be utilized to inflate or deflate the first expandable member 130 and/or the second expandable member 132.

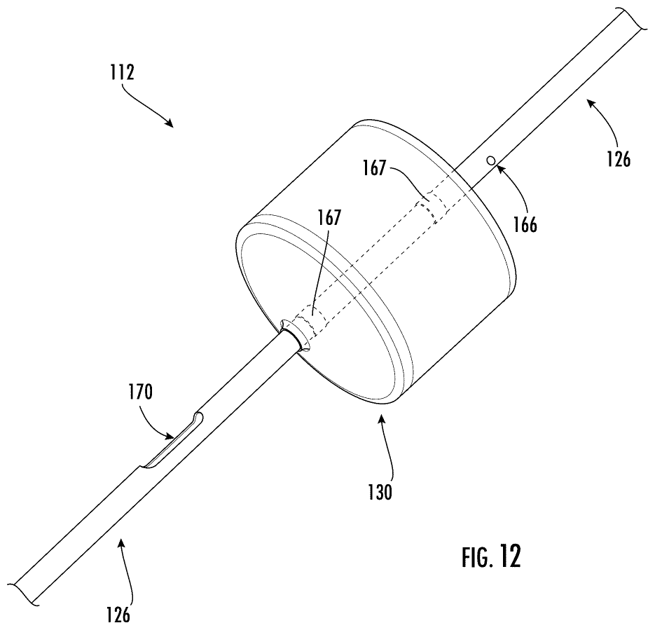

FIG. 12 illustrates a detailed view of a portion of the medical device 112 shown in FIG. 8. For example, FIG. 8 illustrates the first expandable member 130 disposed on the elongate member 126. FIG. 8 illustrates that the medical device 126 may include one or more apertures positioned along the elongate member 126. In some instances, the one or more apertures positioned along the elongate member may be positioned proximate the first expandable member 130. For example, FIG. 12 illustrates that the medical device 126 may include a sensing member aperture 166 positioned proximal to the first expandable member 130. The aperture 166 may extend through the wall of the elongate member 126. Further, it can be appreciated that the aperture 166 may be in fluid communication with the sensing member lumen 140 described herein. It is contemplated that the shape of the sensing member aperture 166 may be square, triangular, rectangular, ovular, polygonal, combinations thereof or any other suitable geometric shape. FIG. 12 further illustrates that the medical device 126 may include one or more marker bands 167 positioned underneath the first expandable member 130 to aid in placement of the first expandable member 130 at the target treatment site.

Further, as described herein, in some examples the sensing member aperture 166 described herein may each extend through the wall of the elongate member 26. However, in other examples it is contemplated that the medical device 12 may include a membrane which extends along the outer surface of the elongate member 26, whereby the membrane extends over the sensing member aperture 166. In these examples, it can be appreciated that a membrane covering the sensing member aperture 166 may be substantially flush with the outer surface of the elongate member 26. Further, the membrane extending across the sensing member aperture 166 may permit the sensing member lumen 156 in fluid communication with the sensing member aperture 166 to be filled with fluid (e.g., the membrane may maintain the fluid within a fluid column defined by the sensing member lumen), whereby the fluid filled lumen may communicate with a pressure sensor of the medical device system 12. It can be appreciated that a change in force occurring along the membrane of the sensing member aperture 166 may be transmitted through its respective fluid filled sensing member lumen 156 to a pressure sensor positioned in the medical device system 10. The pressure sensor may then send a signal to the processor 16 of the control system 14 in response to a change in pressure occurring at the membrane of the sensing member aperture 166.

FIG. 12 further illustrates that the medical device 126 may include a working channel aperture 170 positioned distal to the first expandable member 130. The aperture 170 may extend through the wall of the elongate member 126. Further, it can be appreciated that the aperture 170 may be in fluid communication with the working channel 148 described herein. Accordingly, it can be further appreciated that the working channel aperture 170 may permit an auxiliary medical device (e.g., diagnostic catheter, thermodilution catheter, etc.) which has been inserted into the hub 128 and passed through the working channel 148 to pass through the wall of the elongate member 126, whereby the auxiliary medical device may extend away from the medical device 126 to access other portions of a patient's anatomy. In some instances, a thermodilution catheter may be inserted into the hub 128, through the working channel 148, through the working channel aperture 170, and extend away from the medical device 126 to access the right atrium and/or the pulmonary artery of a patient. It is contemplated that other auxiliary medical devices (in addition to a thermodilution catheter) may be passed through the working channel 148 to access portions of a patient's anatomy.

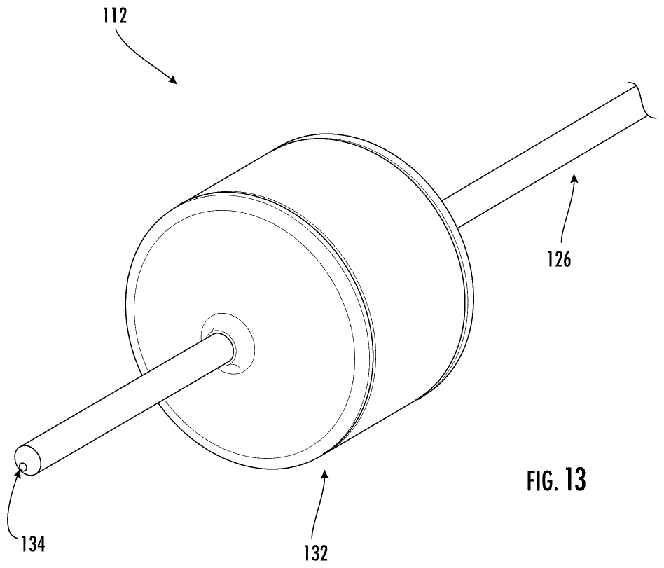

FIG. 13 illustrates a detailed view of a portion of the medical device 112 shown in FIG. 8. For example, FIG. 13 illustrates the second expandable member 132 disposed on the elongate member 126. FIG. 13 illustrates the combination guidewire and sensing member lumen 134 extending within the elongate member 126. As discussed herein, the combination guidewire and sensing member lumen 134 may permit both a guidewire and a sensing member to extend therethrough.

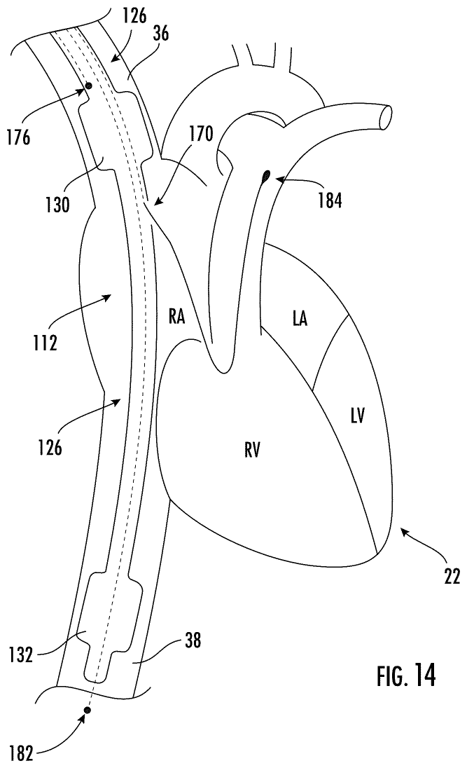

FIG. 14 illustrates the medical device 112 after having been advanced to a position a target treatment site adjacent the heart 22 of a patient 24. The medical device 112 may be tracked over a guidewire extending through the combination guidewire and sensing member lumen 134 described herein. After being advanced proximate the target treatment site as shown in FIG. 14, the guidewire may be removed whereby a sensing member may be advanced through the combination guidewire and sensing member lumen 134. FIG. 14 further illustrates the first expandable member 130 positioned in the superior vena cava 36 and the second expandable member 132 positioned in the inferior vena cava 138. While FIG. 14 illustrates that second expandable member 132 positioned in the inferior vena cava, in other examples it is contemplated that the second expandable member 132 may be positioned proximate the renal arteries or renal veins. Further, FIG. 14 illustrates an auxiliary medical device 184 (e.g., diagnostic medical device, thermodilution catheter, auxiliary sensing member, etc.) extending within the working channel 148, passing through the working channel aperture 170, extending through the right atrium (RA) and into the pulmonary artery (PA) of the patient.

As discussed herein, the elongate member 126 may include one or more individual lumens extending therein. For example, it can be appreciated that the elongate member 126 may include a first inflation lumen 137 (shown in FIG. 9) which may extend from the first expandable member 130 and be in fluid communication with the pump 18 (which, in turn, may be in fluid communication with the saline reservoir 20). Additionally, as discussed herein, the elongate member 126 may include a second inflation lumen 138 (shown in FIG. 9) which may extend from the second expandable member 132 and be in fluid communication with the pump 18 (which, in turn, may be in fluid communication with the saline reservoir 20).

Additionally, FIG. 14 illustrates that the elongate member 126 may include one or more additional lumens (in addition to the inflation lumens described above), which may be configured to permit a sensing member to extend therein. For example, FIG. 14 illustrates a sensing member 176 extending within the sensing member lumen 140, whereby the distal end of the sensing member 176 may be positioned proximal of the first expandable member 130. It can be appreciated from FIG. 14 that, when positioned proximate a target treatment site, the distal end of the sensing member 176 may align with the sensing member aperture 166 described herein. Further, FIG. 14 illustrates a sensing member 78 extending within the combination guidewire and sensing member lumen 134, whereby the distal end of the sensing member 182 may extend out of the distal end of the elongate member 126 such that the distal end of the sensing member 182 is positioned distal of the second expandable member 132.

FIG. 14 illustrates the sensing member 176 positioned proximate the first expandable member 130 in the superior vena cava 36. FIG. 14 further illustrates the sensing members 182 positioned distal to the second expandable member 132 in the inferior vena cava 38. In some examples, the sensing members 176, 182 may all be configured to sense a change in one or more physiological parameters/characteristics occurring in a patient 24. Specifically, in some examples, the sensing members 176, 182 may include pressure sensing capabilities. In other words, the sensing members 176, 182 may include a pressure sensor designed to measure the central venous pressure in the superior vena cava 36, the sensing members 176, 182 may include a pressure sensor designed to measure the inferior venous pressure in the inferior vena cava 38.

It can be appreciated that any of the sensing members 176, 182 may include a variety of different configurations. For example, in some instances, the sensing members 176, 182 may include a pressure sensing wire which is integrated into the control system 14. In other examples, the sensing members 176, 182 may an invasive blood pressure (IBP sensor). For example, in some instances, each of the sensing members may include a microelectricalmechanical (MEMS) sensor coupled to an elongated wire. The MEMS pressure sensor may be able to detect and respond rapidly to very small changes in blood pressure. Further, a MEMS pressure sensor may be able to transmit a signal to the processor 16 indicating there has been a change in blood pressure in the region in which the MEMS sensor is disposed (e.g., the superior vena cava, inferior vena cava, or the adjacent the right atrium).

In other examples, each of the sensing members 176, 182 may include a pressure sensing catheter, whereby the catheter includes a fluid-filled lumen, the distal end of which may be open to the surrounding environment. Further, a change in pressure in the area surrounding the distal end of the fluid-filled catheter may cause the fluid within the catheter to shift. This shifting of the fluid within the fluid-filled catheter may be sensed by the processor 16.

Additionally, in yet other examples, each of the sensing members 176, 182 may include a fiber optic pressure sensing catheter, whereby the fiber optic pressure sensing catheter includes a fiber optic pressure sensor. The fiber optic pressure sensor may sense a change in blood pressure (in areas adjacent to the sensor) based on a change in the light intensity surrounding the sensor. This change in light intensity may be sensed by the processor 16. In any of the examples discussed herein, the sensing members may include a variety of sensors. For example, in addition to the sensors discussed above, the sensing members disclosed herein may include piezo-resistive sensors, piezo-capacitive sensors, pressure sensors, flow sensors, accelerometers, temperature sensors, or the like.

As discussed herein, if the pumping action of the heart 22 is compromised due to a weakened left ventricle (for example), blood may begin to back up therein. Further, blood backing up in the left ventricle may result in blood backing up in the left atrium. Further yet, blood backing up in the left atrium may result in blood backing up within the pulmonary veins, the lungs and the pulmonary arteries. Blood backing up in the pulmonary arteries may further result in blood backing up the right ventricle which, over time, results in the backing up of blood (and increased blood pressure) in the right atrium and areas immediately adjacent to the right atrium (e.g., the superior vena cava, the inferior vena cava).

Further, it can be appreciated that as the blood backs up into the right atrium, additional blood may continue to flow into the right atrium from the superior vena cava and the inferior vena cava. Therefore, to mediate the adverse effects caused by the backing up of blood into the right atrium (e.g., the increase in blood volume and blood pressure within the right atrium), it may be desirable to temporarily occlude (fully or partially) the superior vena cava and/or inferior vena cava.

Therefore, in some examples, one or more of the sensing members 176, 182 and/or auxiliary medical device 184 may sense a change in a physiological parameter (e.g., a change in blood pressure, cardiac output, stroke volume, blood flow, HR, MAP, etc.) in the areas surrounding the superior vena cava 36, the inferior vena cava 38, the right atrium and/or pulmonary artery. Further, it can be appreciated that any sensor sensing a change in the parameter may transmit a signal to the processor 16. The processor 16 may include a memory and/or an algorithm which is designed to measure, interpret, process, analyze, compute, evaluate, etc. the signal received from the one or more sensing members 176, 182 and/or auxiliary medical device 184. Further yet, the algorithm may process the signal received from the one or more sensing members 176, 182 and/or the auxiliary medical device 184 and, if necessary, communicate with the pump 18 to fill or evacuate fluid from the first expandable member 130 and/or the second expandable member 132. As discussed above, the pump 18 may draw fluid from the saline reservoir 20 to expand the first expandable member 130 and/or the second expandable member 132.

It can be appreciated that when the sensing members 176, 182 and/or the auxiliary medical device 184 may sense a change in a physiological parameter (e.g., a change in blood pressure, cardiac output, stroke volume, blood flow, HR, MAP, etc.) in the areas surrounding the superior vena cava 36, the inferior vena cava 38, the right atrium and/or pulmonary artery expanding the first expandable member 130 and/or the second expandable member 132 may restrict the flow of blood into the areas surrounding the right atrium. Restricting the flow of blood into the areas surrounding the right atrium may allow excess blood which has built up in the right atrium to vacate (or partially vacate) the right atrium, thereby reducing the blood pressure built up in the heart 22.

In some instances, it may not be necessary for the system 10 to expand the first expandable member 130 and/or the second expandable member 132 to a point in which the expandable members 130, 132 completely occlude the superior vena cava 36 and the inferior vena cava 38, respectively. Rather, in some instances the processor 16 may process the signals received from one or more of the sensing members the sensing members 176, 182 and/or the auxiliary medical device 184 and only partially occlude the superior vena cava 36 and/or the inferior vena cava 38. In other instances, the processor 16 may process the signals received from one or more of the sensing members the sensing members 176, 182 and/or the auxiliary medical device 184 and fully occlude the superior vena cava 36 or the inferior vena cava 38 while leaving the other of the superior vena cava 36 and the inferior vena cava 38 open or only partially occluded. It can be appreciated that the processor 16 may include an algorithm which is designed to analyze varying physiological parameters occurring in the superior vena cava, the inferior vena cava, the right atrium and/or the pulmonary artery to determine the degree to which either the first expandable member 130 and/or the second expandable member 132 should be occluded (if at all).

It is noted that the physiological parameters discussed above which may be processed by the processor 16 to assess the degree to which the first expandable member 130 and/or the second expandable member 132 should be occluded is not limited to merely the blood pressure in the superior vena cava 36, the inferior vena cava 38, the right atrium and/or the pulmonary artery. Rather, the processor 16 may assess blood flow, blood pressure, cardiac output, stroke volume, HR, MAP, motion of the inferior vena cava, motion of the superior vena cava, respiration cycles, volume in the inferior vena cava, volume in the superior vena cava or the like.

Further, in some instances the algorithm utilized by the processor 16 may include an assessment of the blood pressure readings in the superior vena cava 36, the inferior vena cava 38, the right atrium and/or the pulmonary artery for a particular patient, whereby the algorithm may further utilize a look-up table or artificial intelligence algorithm to determine the degree to which either the first expandable member 130 and/or the second expandable member 132 should be occluded for that particular patient at a given time point.

Further yet, in some examples the control system 14 may also be coupled to a sensor which is providing a patient's ECG to the processor 16. In that case, the cardiac cycle timing (e.g., the occlusion duty cycle of may be timed to features of the ECG) of a particular patient may be utilized to determine the degree to which either the first expandable member 130 and/or the second expandable member 132 should be occluded for that particular patient at a given time point.

As discussed above, the timing of the inflation or evacuation of the first expandable member 130 may be different that the timing of inflation or evacuation of the second expandable member 132. In other words, the inflation or evacuation of the first expandable member 130 may not be in sync with the inflation or evacuation of the second expandable member 132. Permitting the inflation or evacuation of the first expandable member 130 to be out of sync with the second expandable member 132 may allow a minimal impact on cerebral blood flow without raising renal or hepatic venous pressures and reducing cardiac workload while maintaining overall mean arterial pressures. The inflation or evacuation of the first expandable member 230 and the second expandable member 232 may be actuated intermittently, continuously, synchronously, asynchronously, individually or automatically based on various physiological parameters (e.g., blood pressure, cardiac output, stroke volume, arterial pressure, venous pressure, superior vena cava pressure, inferior vena cava pressure, right atrial pressure, pulmonary artery pressure, mean arterial pressure, heart rate, renal artery pressure, renal vein pressure).

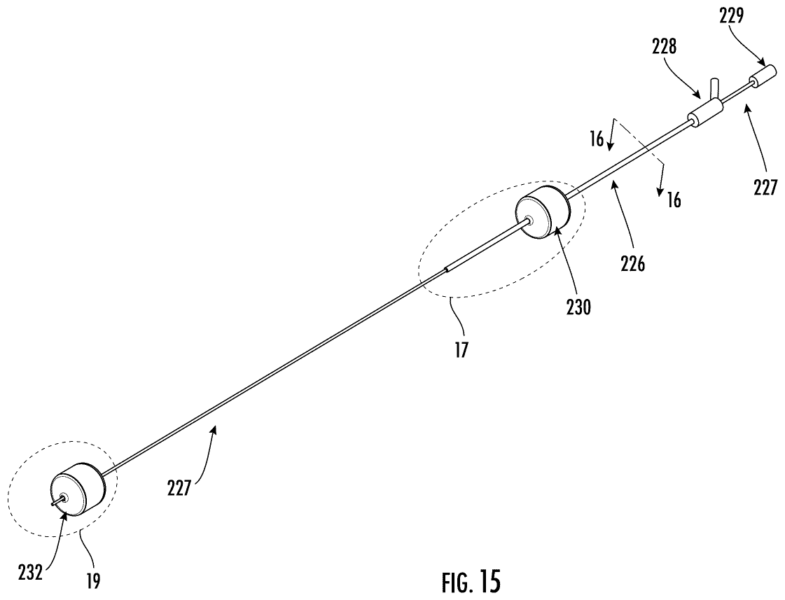

FIG. 15 illustrates another example medical device 212 which may be utilized with the system 10 described herein. The medical device 212 shown in FIG. 15 may be similar in form and function to other medical devices described herein. For example, the medical device 212 may include a first expandable member 230 disposed on a first elongate member 226 and a second expandable member 232 disposed on a second elongate member 227. The second expandable member 232 may be positioned distal to the first expandable member 230. In some instances, each of the first expandable member 230 and the second expandable member 232 may be referred to as an expandable medical balloon. The first expandable member 230 may include a distal end and a proximal end. Both the distal and proximal end of the first expandable member 230 may be coupled to the elongate member 226. Further, the second expandable member 232 may include a distal end and a proximal end. Both the distal and proximal end of the second expandable member 232 may be coupled to the elongate member 227.

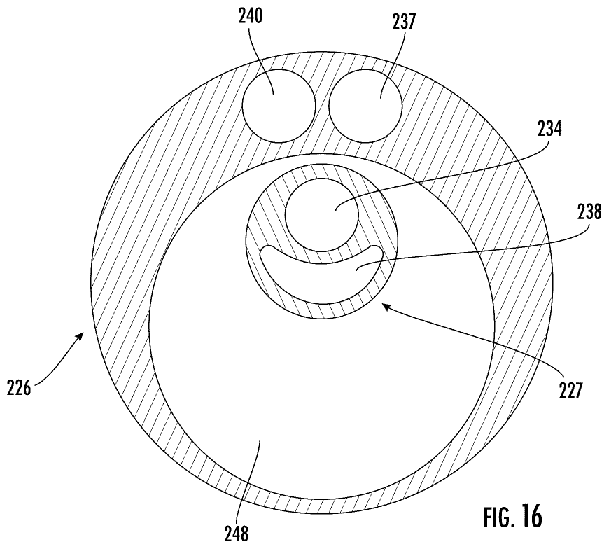

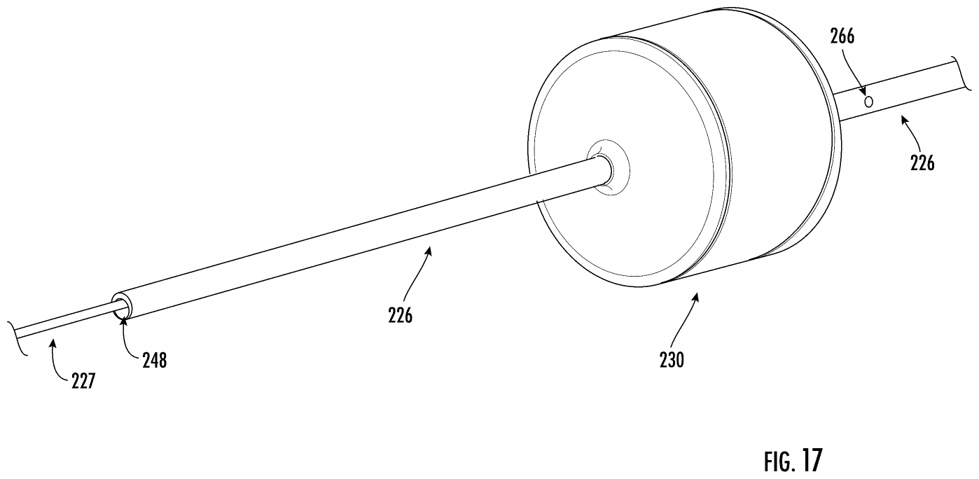

FIG. 15 further illustrates that the first elongate member 226 and the second elongate member 227 may be separate, distinct shafts whereby the second elongate member 227 may extend within a lumen of the first elongate shaft 226 (FIGS. 16-17 illustrate the second elongate member 227 extending within a lumen of the first elongate member 226). Accordingly, it can be appreciated that the second elongate member 227 may translate (e.g., shift, move, telescope) relative to the first elongate member 226. In other words, a user may adjust the spacing between the first expandable member 230 and the second expandable member 232 by sliding the second elongate member 227 within the lumen of the first elongate member 226. In some examples, the distal end of the first expandable member 30 may be spaced from the proximal end of the second expandable member 32 about 2″ to about 25″, or about 8″ to about 22″, or about 12″ to about 18″ or about 15″.

FIG. 15 further illustrates that the proximal end of each of the first elongate member 226 and the second elongate member 227 may be coupled to a hub. For example, FIG. 15 illustrates that the proximal end of the first elongate member 226 may be coupled to a first hub 228 and the proximal end of the second elongate member 227 may be coupled to a second hub 229. Each of the hubs 228, 229 may include one or more ports which may be in fluid communication with the control system 14 and/or the saline reservoir 20. Additionally, each of the hubs 228, 229 may include one or more ports which may permit one or more medical devices (e.g., auxiliary medical devices) to be inserted therein.

It can be further appreciated that the hubs 228, 229 may be utilized to translate the second expandable member 232 relative to the first expandable member 230. In other words, moving the hub 229 relative to the hub 248 may translate the second expandable member 232 relative to the first expandable member 230, thereby permitting a user to adjust the distance (e.g., increasing or decreasing the spacing) between the second expandable member 232 relative and the first expandable member 230. In some examples, the second expandable member 232 may be able to extend from about 2″ to about 25″, or about 8″ to about 22″, or about 12″ to about 18″ or about 15″ away from the first expandable member 230. It can be appreciated that a user may manipulate the hubs 228, 229 such that the first expandable member 230 is positioned in the superior vena cava and the second expandable member 232 is positioned in the inferior vena cava. In other examples, a user may manipulate the hubs 228, 229 such that the first expandable member 230 is positioned in the superior vena cava and the second expandable member 232 is positioned proximate the renal veins.

In some examples, the elongate member 227 may pass through the expandable member 232 and extend distally beyond the expandable member 232 to define a distal tip of the medical device 21. In other examples, the medical device 212 may include a separate tip member which is attached (e.g., bonded) to the distal waist of the expandable member 232 and/or also (e.g., bonded) to the elongate member 227. In other words, in some examples, the elongate member 227 itself may form the distal tip of the medical device 212, however, in other examples, a separate tip member may be attached to the expandable member 232, the elongate member 227 or both the expandable member 232 and the elongate member 227 to form the distal tip of the medical device 212.

FIG. 16 illustrates a cross-section of the medical device 212 taken along line 16-16 of FIG. 15. As described herein, FIG. 16 illustrates the second elongate member 227 extending within a working channel 248 of the first elongate member 226. In some examples, the working channel 248 may have a diameter of about 0.090″ to about 0.145″, or about 0.100″ to about 0.135″, or about 0.115″ to about 0.125″, or about 0.116″ to about 0.120″, or about 0.118″.

Additionally, FIG. 16 illustrates that the second elongate member 227 may include a combination guidewire and sensing member lumen 234. The combination guidewire and sensing member lumen 234 may extend from the proximal end of the second elongate member 227 to the distal end of the second elongate member 227. In some examples, the combination sensing and guidewire lumen 234 may have a diameter of about 0.010″ to about 0.045″, or about 0.015″ to about 0.035″, or about 0.019″ to about 0.029″, or about 0.022″ to about 0.027″, or about 0.025″.

It can be appreciated that, during a medical procedure, the distal end of the elongate member 227 may be tracked over a guidewire, whereby the guidewire may pass through the combination guidewire and sensing member lumen 234 and exit the medical device 212 through a port of the hub 249. In other words, the combination guidewire and sensing member lumen 234 may permit the medical device 212 to be tracked over a guidewire (previously positioned within a patient) to a treatment site (e.g., the heart) within the patient. As will be described in greater detail below, after the medical device 212 is tracked over a guidewire (previously positioned within a patient) to a treatment site, the guidewire may be removed from the patient, whereby the combination guidewire and sensing member lumen 234 may be utilized to pass a sensing member therethrough to a position adjacent the treatment site.

FIG. 16 further illustrates that the first elongate member 226 may include a first inflation lumen 237 which may be in fluid communication with the pump 18 (which, in turn, may be in fluid communication with the saline reservoir 20). In some examples, the first inflation lumen 237 may be in fluid communication with the first expandable member 230. It can be appreciated that the saline from the saline reservoir 20 may be passed through a first inflation port) on the hub 228, through the first inflation lumen 237 and into the first expandable member 230 to expand or deflate the first expandable member 230. In some examples, the first inflation lumen 237 may have a diameter of about 0.010″ to about 0.045″, or about 0.015″ to about 0.035″, or about 0.019″ to about 0.029″, or about 0.022″ to about 0.027″, or about 0.025″.

In some examples, the wall thickness between adjacent lumens 237, 240 of the elongate member 126 illustrated in FIG. 9 may be about 0.0015″ to about 0.0085″, or about 0.0025″ to about 0.0075″, or about 0.0035″ to about 0.0065″, or about 0.0045″ to about 0.0055″, or about 0.0050″.

FIG. 16 further illustrates that the second elongate member 227 may include a second inflation lumen 238 which may be in fluid communication with the pump 18 (which, in turn, may be in fluid communication with the saline reservoir 20). In some examples, the second inflation lumen 238 may be in fluid communication with the second expandable member 232. It can be appreciated that the saline from the saline reservoir 20 may be passed through a second inflation port on the hub 229, through the second inflation lumen 238 and into the second expandable member 232 to expand or deflate the second expandable member 232. As illustrated in FIG. 16, the second inflation lumen 238 may include a crescent shape (e.g., a curved shape that is wider in the middle than at its ends). This is not intended to be limiting. It is contemplated that the second inflation lumen 238 may include a square, triangular, ovular, rectangular, polygonal, combinations thereof or any other suitable geometric shape.

FIG. 16 further illustrates that the first elongate member 226 may include a lumen 240 and the combination guidewire and sensing lumen 234 which are each configured to permit a sensing member to extend therein. For example, FIG. 16 illustrates a sensing member lumen 240, which may be designed to permit a sensing member to extend through the elongate member 226 to a position whereby the sensing member is proximal to the first expandable balloon 230 and within the superior vena cava. In some examples, the sensing member lumen 240 may have a diameter of about 0.010″ to about 0.045″, or about 0.015″ to about 0.035″, or about 0.019″ to about 0.029″, or about 0.022″ to about 0.027″, or about 0.025″. As discussed herein, FIG. 16 further illustrates the combination guidewire and sensing member lumen 234, which may be designed to permit a sensing member to extend through the second elongate member 227 to a position whereby the sensing member extends out of the distal end of the second elongate member 227 to a position distal to the second expandable balloon 232 and within the inferior vena cava.

FIG. 17 illustrates a detailed view of a portion of the medical device 212 shown in FIG. 15. For example, FIG. 17 illustrates the first expandable member 230 disposed on the elongate member 226. FIG. 17 illustrates that the medical device 226 may include a sensing member aperture 266 positioned along the elongate member 226. In some instances, the sensing member aperture 266 positioned along the elongate member may be positioned adjacent to the first expandable member 230. The aperture 266 may extend through the wall of the elongate member 226. Further, it can be appreciated that the aperture 266 may be in fluid communication with the sensing member lumen 240 described herein. It is contemplated that the shape of the sensing member aperture 266 may be square, triangular, rectangular, ovular, polygonal, combinations thereof or any other suitable geometric shape.

Further, as described herein, in some examples the sensing member aperture 266 described herein may each extend through the wall of the elongate member 226. However, in other examples it is contemplated that the medical device 212 may include a membrane which extends along the outer surface of the elongate member 226, whereby the membrane extends over the sensing member aperture 266. In these examples, it can be appreciated that a membrane covering the sensing member aperture 266 may be substantially flush with the outer surface of the elongate member 226. Further, the membrane extending across the sensing member aperture 266 may permit the sensing member lumen 240 in fluid communication with the sensing member aperture 266 to be filled with fluid (e.g., the membrane may maintain the fluid within a fluid column defined by the sensing member lumen), whereby the fluid filled lumen may communicate with a pressure sensor of the medical device system 212. It can be appreciated that a change in force occurring along the membrane of the sensing member aperture 266 may be transmitted through its respective fluid filled sensing member lumen 240 to a pressure sensor positioned in the medical device system 10. The pressure sensor may then send a signal to the processor 16 of the control system 14 in response to a change in pressure occurring at the membrane of the sensing member aperture 266.

FIG. 17 further illustrates a detailed view of FIG. 15 showing the distal end region of the medical device 212. As described herein, FIG. 17 illustrates the second elongate member 227 extending within the working channel 248 of the first elongate member 226. Additionally, it can be further appreciated that the working channel 248 may be designed to permit an auxiliary medical device (e.g., thermodilution catheter) to pass through the hub 228 and the working channel 248 whereby the auxiliary medical device may exit the working channel 248 at the distal end of the first elongate member 226. After exiting the working channel 248, the auxiliary medical device may extend into the right atrium and/or the pulmonary artery of a patient.

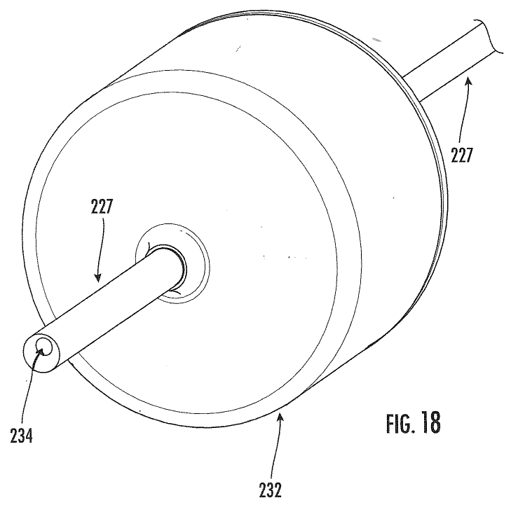

FIG. 18 illustrates a detailed view of a portion of the medical device 212 shown in FIG. 15. For example, FIG. 18 illustrates the second expandable member 232 disposed on the elongate member 227. FIG. 18 illustrates the combination guidewire and sensing member lumen 234 extending within the elongate member 227. As discussed herein, the combination guidewire and sensing member lumen 234 may permit both a guidewire and a sensing member to extend therethrough.

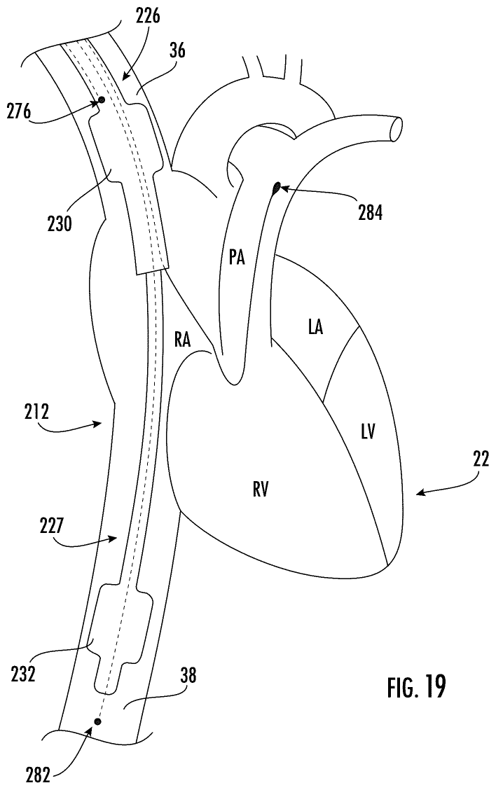

FIG. 19 illustrates the medical device 212 after having been advanced to a position proximate a target treatment site adjacent the heart 22 of a patient 24. The medical device 212 may be tracked over a guidewire extending through the combination guidewire and sensing member lumen 234 described herein. After being advanced to the target treatment site as shown in FIG. 19, the guidewire may be removed from the combination guidewire and sensing member lumen 234 whereby a sensing member may be advanced through the combination guidewire and sensing member lumen 234. FIG. 19 further illustrates the first expandable member 230 positioned in the superior vena cava 36 and the second expandable member 232 positioned in the inferior vena cava 38. While FIG. 19 illustrates that second expandable member 232 positioned in the inferior vena cava, in other examples it is contemplated that the second expandable member 232 may be positioned proximate the renal arteries or renal veins. Further, FIG. 19 illustrates an auxiliary medical device 284 (e.g., diagnostic medical device, thermodilution catheter, auxiliary sensing member, etc.) extending within the working channel 248, out the distal end of the first elongate member 226, extending through the right atrium (RA) and into the pulmonary artery (PA) of the patient.

As discussed herein, the elongate member 226 may include one or more individual lumens extending therein. For example, it can be appreciated that the elongate member 226 may include a first inflation lumen 237 (shown in FIG. 16) which may extend from the first expandable member 230 and be in fluid communication with the pump 18 (which, in turn, may be in fluid communication with the saline reservoir 20). Additionally, as discussed herein, the elongate member 227 may include an inflation lumen 286 (shown in FIG. 16) which may extend from the second expandable member 232 and be in fluid communication with the pump 18 (which, in turn, may be in fluid communication with the saline reservoir 20).

Additionally, FIG. 19 illustrates that the elongate member 226 may include one or more additional lumens (in addition to the inflation lumens described above), which may be configured to permit a sensing member to extend therein. For example, FIG. 19 illustrates a sensing member 276 extending within the sensing member lumen 240, whereby the distal end of the sensing member 276 may be positioned proximal of the first expandable member 230. It can be appreciated from FIG. 19 that the distal end of the sensing member 276 may align with a sensing member aperture 266 described herein. Further, FIG. 19 illustrates a sensing member 282 extending within the combination guidewire and sensing member lumen 234, whereby the distal end of the sensing member 282 may extend out of the distal end of the elongate member 227 such that the distal end of the sensing member 282 is positioned distal of the second expandable member 232.

FIG. 19 illustrates the sensing member 276 positioned adjacent the first expandable member 230 in the superior vena cava 36. FIG. 19 further illustrates the sensing member 282 positioned distal to the second expandable member 232 in the inferior vena cava 38. In some examples, the sensing members 276, 282 may be configured to sense a change in one or more physiological parameters/characteristics occurring in a patient 24. Specifically, in some examples, the sensing members 276, 282 may include pressure sensing capabilities. In other words, the sensing members 276, 282 may include a pressure sensor designed to measure the central venous pressure in the superior vena cava 36, the sensing members 276, 282 may include a pressure sensor designed to measure the inferior venous pressure in the inferior vena cava 38.

It can be appreciated that any of the sensing members 276, 282 may include a variety of different configurations. For example, in some instances, the sensing members 276, 282 may include a pressure sensing wire which is integrated into the control system 14. In other examples, the sensing members 276, 282 may include an invasive blood pressure (IBP sensor). For example, in some instances, each of the sensing members may include a microelectricalmechanical (MEMS) sensor coupled to an elongated wire. The MEMS pressure sensor may be able to detect and respond rapidly to very small changes in blood pressure. Further, a MEMS pressure sensor may be able to transmit a signal to the processor 16 indicating there has been a change in blood pressure in the region in which the MEMS sensor is disposed (e.g., the superior vena cava, inferior vena cava, or the adjacent the right atrium).

In other examples, each of the sensing members 276, 282 may include a pressure sensing catheter, whereby the catheter includes a fluid-filled lumen, the distal end of which may be open to the surrounding environment. Further, a change in pressure in the area surrounding the distal end of the fluid-filled catheter may cause the fluid within the catheter to shift. This shifting of the fluid within the fluid-filled catheter may be sensed by the processor 16.

Additionally, in yet other examples, each of the sensing members 276, 282 may include a fiber optic pressure sensing catheter, whereby the fiber optic pressure sensing catheter includes a fiber optic pressure sensor. The fiber optic pressure sensor may sense a change in blood pressure (in areas adjacent to the sensor) based on a change in the light intensity surrounding the sensor. This change in light intensity may be sensed by the processor 16. In any of the examples discussed herein, the sensing members may include a variety of sensors. For example, in addition to the sensors discussed above, the sensing members disclosed herein may include piezo-resistive sensors, piezo-capacitive sensors, pressure sensors, flow sensors, accelerometers, temperature sensors, or the like.

As discussed herein, if the pumping action of the heart 22 is compromised due to a weakened left ventricle (for example), blood may begin to back up therein. Further, blood backing up in the left ventricle may result in blood backing up in the left atrium. Further yet, blood backing up in the left atrium may result in blood backing up within the pulmonary veins, the lungs and the pulmonary arteries. Blood backing up in the pulmonary arteries may further result in blood backing up the right ventricle which, over time, results in the backing up of blood (and increased blood pressure) in the right atrium and areas immediately adjacent to the right atrium (e.g., the superior vena cava, the inferior vena cava).

Further, it can be appreciated that as the blood backs up into the right atrium, additional blood may continue to flow into the right atrium from the superior vena cava and the inferior vena cava. Therefore, to mediate the adverse effects caused by the backing up of blood into the right atrium (e.g., the increase in blood volume and blood pressure within the right atrium), it may be desirable to temporarily occlude (fully or partially) the superior vena cava and/or inferior vena cava.

Therefore, in some examples, one or more of the sensing members 276, 282 and/or auxiliary medical device 284 may sense a change in a physiological parameter (e.g., a change in blood pressure, cardiac output, stroke volume, blood flow, HR, MAP, etc.) in the areas surrounding the superior vena cava 36, the inferior vena cava 38, the right atrium and/or pulmonary artery. Further, it can be appreciated that any sensor sensing a change in the parameter may transmit a signal to the processor 16. The processor 16 may include a memory and/or an algorithm which is designed to measure, interpret, process, analyze, compute, evaluate, etc. the signal received from the one or more sensing members 276, 282 and/or auxiliary medical device 284. Further yet, the algorithm may process the signal received from the one or more sensing members 276, 282 and/or the auxiliary medical device 284 and, if necessary, communicate with the pump 18 to fill or evacuate fluid from the first expandable member 230 and/or the second expandable member 232. As discussed above, the pump 18 may draw fluid from the saline reservoir 20 to expand the first expandable member 230 and/or the second expandable member 232.

It can be appreciated that when the sensing members 276, 282 and/or the auxiliary medical device 284 may sense a change in a physiological parameter (e.g., a change in blood pressure, cardiac output, stroke volume, blood flow, HR, MAP, etc.) in the areas surrounding the superior vena cava 36, the inferior vena cava 38, the right atrium and/or pulmonary artery expanding the first expandable member 230 and/or the second expandable member 232 may restrict the flow of blood into the areas surrounding the right atrium. Restricting the flow of blood into the areas surrounding the right atrium may allow excess blood which has built up in the right atrium to vacate (or partially vacate) the right atrium, thereby reducing the blood pressure built up in the heart 22.

In some instances, it may not be necessary for the system 10 to expand the first expandable member 230 and/or the second expandable member 232 to a point in which the expandable members 230, 232 completely occlude the superior vena cava 36 and the inferior vena cava 38, respectively. Rather, in some instances the processor 16 may process the signals received from one or more of the sensing members the sensing members 276, 282 and/or the auxiliary medical device 284 and only partially occlude the superior vena cava 36 and/or the inferior vena cava 38. In other instances, the processor 16 may process the signals received from one or more of the sensing members the sensing members 276, 282 and/or the auxiliary medical device 284 and fully occlude the superior vena cava 36 or the inferior vena cava 38 while leaving the other of the superior vena cava 36 and the inferior vena cava 38 open or only partially occluded. It can be appreciated that the processor 16 may include an algorithm which is designed to analyze varying physiological parameters occurring in the superior vena cava, the inferior vena cava, the right atrium and/or the pulmonary artery to determine the degree to which either the first expandable member 230 and/or the second expandable member 232 should be occluded (if at all).

It is noted that the physiological parameters discussed above which may be processed by the processor 16 to assess the degree to which the first expandable member 230 and/or the second expandable member 232 should be occluded is not limited to merely the blood pressure in the superior vena cava 36, the inferior vena cava 38, the right atrium and/or the pulmonary artery. Rather, the processor 16 may assess blood flow, blood pressure, cardiac output, blood volume, motion of the inferior vena cava, motion of the superior vena cava, respiration cycles, volume in the inferior vena cava, volume in the superior vena cava or the like.

Further, in some instances the algorithm utilized by the processor 16 may include an assessment of the blood pressure readings in the superior vena cava 36, the inferior vena cava 38, the right atrium and/or the pulmonary artery for a particular patient, whereby the algorithm may further utilize a look-up table or artificial intelligence algorithm to determine the degree to which either the first expandable member 230 and/or the second expandable member 232 should be occluded for that particular patient at a given time point.

Further yet, in some examples the control system 14 may also be coupled to a sensor which is providing a patient's ECG to the processor 16. In that case, the cardiac cycle timing (e.g., the occlusion duty cycle of may be timed to features of the ECG) of a particular patient may be utilized to determine the degree to which either the first expandable member 230 and/or the second expandable member 232 should be occluded for that particular patient at a given time point.

As discussed above, the timing of the inflation or evacuation of the first expandable member 230 may be different that the timing of inflation or evacuation of the second expandable member 232. In other words, the inflation or evacuation of the first expandable member 230 may not be in sync with the inflation or evacuation of the second expandable member 232. Permitting the inflation or evacuation of the first expandable member 230 to be out of sync with the second expandable member 232 may allow a minimal impact on cerebral blood flow without raising renal or hepatic venous pressures and reducing cardiac workload while maintaining overall mean arterial pressures.

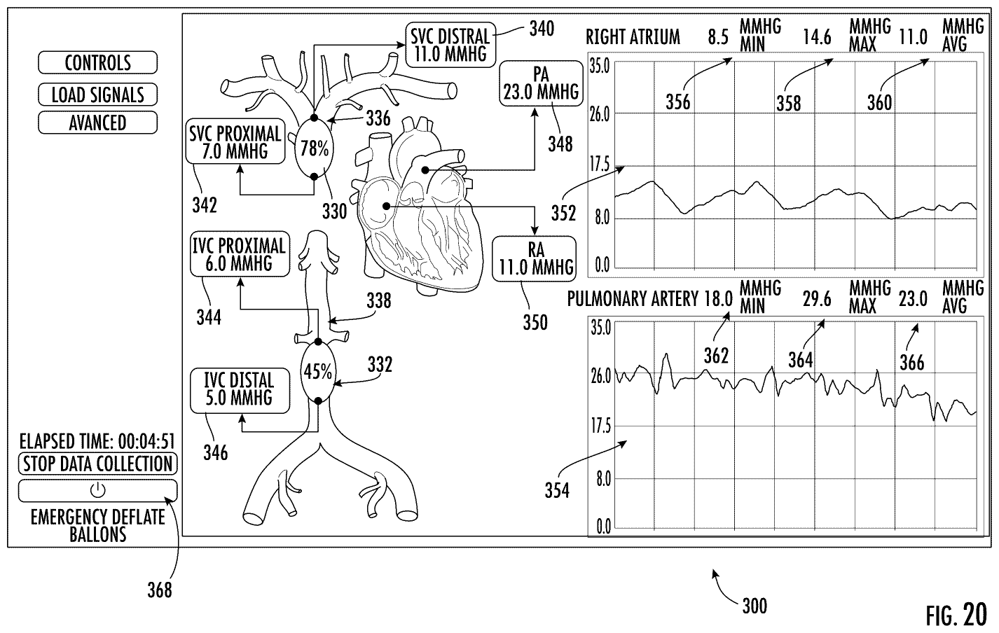

FIGS. 20-22 illustrate example displays 300, 400, 500 which may be utilized with the medical device system 10 described herein. The example displays 300, 400, 500 may represent the display 15 described herein with respect to FIG. 1. In some examples, the displays 300, 400, 500 may include a CRT, LED, 3D display, GUI, or other type of display, for example. The displays 300, 400, 500 may present information relevant to functional and operational parameters of the medical device system 10, circulatory support devices 12, 112, 212 and/or physiological parameters of the patient 24 in a simple format useful to clinicians. For example, the displays 300, 400, 500 may be configured to display graphical information, visual representations, symbols, icons, etc. relevant to a patient's anatomical features, the inflation percentage of the first expandable member and/or the inflation percentage of the second expandable member, advanced hemodynamic metrics received from an auxiliary medical device (e.g., a thermodilution catheter), various physiological parameters including, but not limited to, cardiac output, stroke volume, an arterial pressure, a venous pressure, superior vena cava pressure proximal an occlusive balloon, superior vena cava pressure distal an occlusive balloon, inferior vena cava pressure proximal an occlusive balloon, inferior vena cava pressure distal an occlusive balloon, right atrial pressure, pulmonary artery pressure, mean arterial pressure, heart rate, renal artery pressure, renal vein pressure, etc., a gradient (e.g., increase and/decrease) in the magnitude or differential between any physiological parameter disclosed herein, any physiological parameter measurement derived from a thermodilution catheter, any combination or temporal pattern of signals corresponding to one or more of the physiological parameters listed herein. It can be appreciated that any of the example displays 300, 400, 500 described herein may be designed to display any one or more graphical information representation, visual representation, symbol, icon, etc. described herein in a singular format or in any combination with any other graphical representation, visual representation, symbol, icon, etc. described herein.

Further, additional parameters (e.g., flow through the pump 18) may be derived by processing any combination or temporal pattern of signals corresponding to one or more of the parameters listed herein in a time dependent manner. Further, the displays 300, 400, 500 may indicate if the system 10 is operating normally, if the system 10 has detected a specific issue that may require additional (e.g., non-routine) procedures/adjustments, and/or if the system 10 has detected a specific issue that requires emergency procedures. Furthermore, the displays 300, 400, 500 may include information which conveys that a particular response (e.g., action) is needed. For example, the displays 300, 400, 500 may be able to convey information that the pump 22 has failed and needs to be replaced immediately during a medical procedure. The displays 300, 400, 500 may also be able to convey that the first expandable member and/or the second expandable member need to be further inflated or deflated in response to a change in a patient's anatomical features, the inflation percentage of the first expandable member and/or the inflation percentage of the second expandable member, advanced hemodynamic metrics received from an auxiliary medical device (e.g., a thermodilution catheter), various physiological parameters including, but not limited to, cardiac output, stroke volume, an arterial pressure, a venous pressure, superior vena cava pressure proximal an occlusive balloon, superior vena cava pressure distal an occlusive balloon, inferior vena cava pressure proximal an occlusive balloon, inferior vena cava pressure distal an occlusive balloon, right atrial pressure, pulmonary artery pressure, mean arterial pressure, heart rate, renal artery pressure, renal vein pressure, etc., a gradient (e.g., increase and/decrease) in the magnitude or differential between any physiological parameter disclosed herein, any physiological parameter measurement derived from a thermodilution catheter, any combination or temporal pattern of signals corresponding to one or more of the physiological parameters listed herein.

It can be appreciated that the displays 300, 400, 500 may be designed to provide a simplified, visual information summary designed to easily convey the status of one or more functional, operational, anatomical and/or physiological parameters of the medical device system 10 and/or the patient 16. In some examples, the graphical user interface of the displays 300, 400, 500 may permit a user to select, input and control various functionality of the medical device system 10. For example, the graphical user interface of the displays 300, 400, 500 may permit a user to immediately stop the inflation and/or deflation of the first and/or second expandable members based on feedback received from one or more sensing members which may be represented on the displays 300, 400, 500, for example.

FIG. 20 is a schematic depiction of an example display 300 as discussed herein. FIG. 20 illustrates the display 300 may display (e.g., illuminate) one or more of an example arrangement and the relative position of one or more anatomical symbols (e.g., icons, visual representations, words, signs, visual alerts, etc.), a graphical display of a pressure waveform, a graphical display of physiological parameters (e.g., blood pressure, cardiac output, stroke volume, arterial pressure, venous pressure, superior vena cava pressure, inferior vena cava pressure, right atrial pressure, pulmonary artery pressure, mean arterial pressure, heart rate, renal artery pressure, renal vein pressure). As will be discussed in greater detail below, one or more of the symbols may be illuminated on the display 300 in a variety of different configurations.

As illustrated in FIG. 20, the example display 300 may include an anatomical representation of a patient's superior vena cava 336 and anatomical representation of a patient's inferior vena cava 338. FIG. 20 illustrates that the display 300 may further include a visual representation of a first expandable member 330 positioned in the superior vena cava 336 and a second expandable member 332 positioned in the inferior vena cava 338. It can be appreciated that the first expandable member 330 may represent the first expandable member 30, 130, 230 described herein, while the second expandable member 332 may represent the second expandable member 32, 132, 232 described herein.

FIG. 20 further illustrates that the display 300 may include a visual representation 340 of a physiological parameter (e.g., blood pressure) sensed proximate to an expandable member positioned in the superior vena cava (e.g., a parameter sensed by a sensing member 76, 176, 276 placed proximal an expandable member positioned in the superior vena cava). FIG. 20 further illustrates that the display 300 may include a visual representation 342 of a physiological parameter (e.g., blood pressure) sensed proximate to an expandable member positioned in the superior vena cava (e.g., a parameter sensed by a sensing member 78 placed distal an expandable member positioned in the superior vena cava). FIG. 20 further illustrates that the display 300 may include a visual representation 344 of a physiological parameter (e.g., blood pressure) sensed proximate to an expandable member positioned in the inferior vena cava (e.g., a parameter sensed by a sensing member 80 placed proximal an expandable member positioned in the inferior vena cava). FIG. 20 further illustrates that the display 300 may include a visual representation 346 of a physiological parameter (e.g., blood pressure) sensed proximate to an expandable member positioned in the inferior vena cava (e.g., a parameter sensed by a sensing member 82, 182, 282 placed proximal an expandable member positioned in the inferior vena cava).

Additionally, FIG. 20 illustrates that the display 300 may include one or more visual representations 348, 350 of a physiological parameter and/or hemodynamic data (e.g., right arterial blood pressure, right ventricular pressure, cardiac output, stroke volume, etc.) sensed by a sensing member 84, 184, 284 placed proximate the right atrium and/or the pulmonary artery. As discussed herein, the sensing member 84, 184, 284 may include a thermodilution catheter.

FIG. 20 further illustrates that the display 300 may further include a visual representation 352 which may include a blood pressure waveform, whereby the blood pressure waveform conveys blood pressure values of the right atrium over time. The visual representation 352 may further include a visual representation 356 of the minimum value of the blood pressure of the right atrium over a given time period, a visual representation 358 of the maximum value of the blood pressure of the right atrium over a given time period and a visual representation 360 of the average value of the blood pressure of the right atrium over a given time period.

FIG. 20 further illustrates that the display 300 may further include a visual representation 354 which may include a blood pressure waveform, whereby the blood pressure waveform conveys blood pressure values of the pulmonary artery over time. The visual representation 354 may further include a visual representation 362 of the minimum value of the blood pressure of the pulmonary artery over a given time period, a visual representation 364 of the maximum value of the blood pressure of the pulmonary artery over a given time period and a visual representation 366 of the average value of the blood pressure of the pulmonary artery over a given time period.

FIG. 20 illustrates the visual representations 352, 354 including a blood pressure waveforms of the right atrium and pulmonary artery, respectively. However, this is not intended to be limiting. Rather, it is contemplated that the visual representations 352, 354 may include visual representations of a variety of physiological parameters including, but not limited to cardiac output, stroke volume, arterial pressure, venous pressure, superior vena cava pressure, inferior vena cava pressure, right atrial pressure, pulmonary artery pressure, mean arterial pressure, heart rate, renal artery pressure, renal vein pressure, combinations thereof or any other physiological parameter.

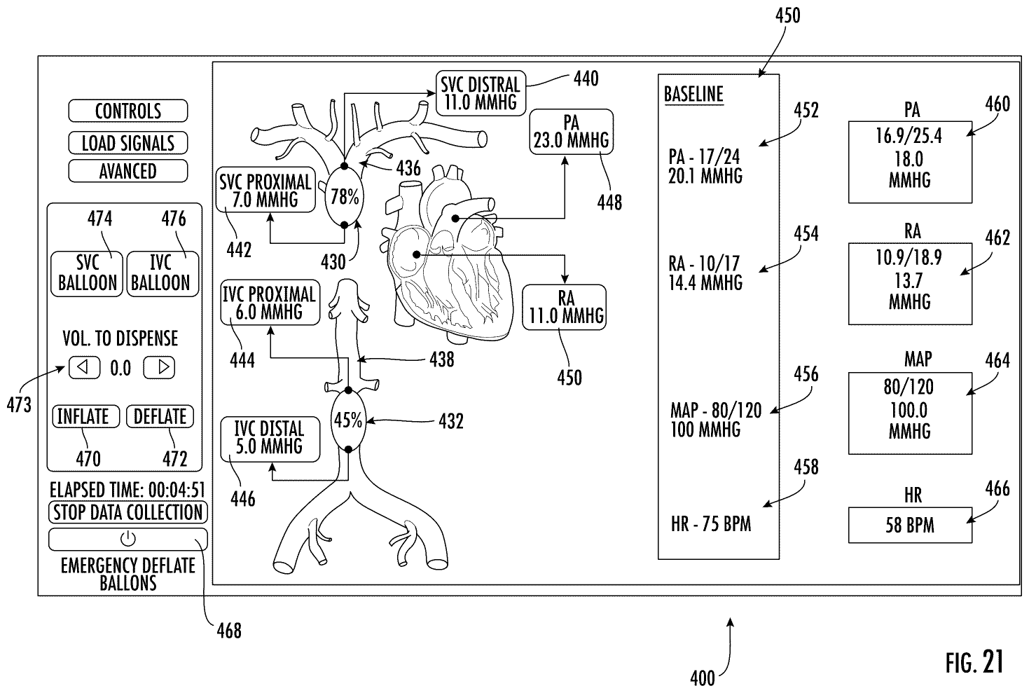

FIG. 21 is a schematic depiction of an example display 400 as discussed herein. FIG. 21 illustrates the display 400 may display (e.g., illuminate) one or more of an example arrangement and the relative position of one or more anatomical symbols (e.g., icons, visual representations, words, signs, visual alerts, etc.) and/or a graphical display of one or more physiological parameters (e.g., blood pressure, cardiac output, stroke volume, arterial pressure, venous pressure, superior vena cava pressure, inferior vena cava pressure, right atrial pressure, pulmonary artery pressure, mean arterial pressure, heart rate, renal artery pressure, renal vein pressure). As will be discussed in greater detail below, one or more of the symbols may be illuminated on the display 400 in a variety of different configurations.

As illustrated in FIG. 21, the example display 400 may include an anatomical representation of a patient's superior vena cava 436 and anatomical representation of a patient's inferior vena cava 438. FIG. 21 illustrates that the display 400 may further include a visual representation of a first expandable member 430 positioned in the superior vena cava 436 and a second expandable member 432 positioned in the inferior vena cava 438. It can be appreciated that the first expandable member 430 may represent the first expandable member 30, 130, 230 described herein, while the second expandable member 432 may represent the second expandable member 32, 132, 232 described herein.

FIG. 21 further illustrates that the display 400 may include a visual representation 440 of a physiological parameter (e.g., blood pressure) sensed proximate to an expandable member positioned in the superior vena cava (e.g., a parameter sensed by a sensing member 76, 176, 276 placed proximal an expandable member positioned in the superior vena cava). FIG. 21 further illustrates that the display 400 may include a visual representation 442 of a physiological parameter (e.g., blood pressure) sensed proximate to an expandable member positioned in the superior vena cava (e.g., a parameter sensed by a sensing member 78 placed distal an expandable member positioned in the superior vena cava). FIG. 21 further illustrates that the display 400 may include a visual representation 444 of a physiological parameter (e.g., blood pressure) sensed proximate to an expandable member positioned in the inferior vena cava (e.g., a parameter sensed by a sensing member 80 placed proximal an expandable member positioned in the inferior vena cava). FIG. 21 further illustrates that the display 400 may include a visual representation 446 of a physiological parameter (e.g., blood pressure) sensed proximate to an expandable member positioned in the inferior vena cava (e.g., a parameter sensed by a sensing member 82, 182, 282 placed proximal an expandable member positioned in the inferior vena cava).

Additionally, FIG. 21 illustrates that the display 400 may include a visual representation 450 of “baseline” values one or more physiological parameters and/or hemodynamic data (e.g., right arterial blood pressure, right ventricular pressure, cardiac output, stroke volume, etc.) of a patient. For example, FIG. 21 illustrates baseline values for the pressure in the pulmonary artery 452, pressure in the right atrium 454, mean arterial pressure 456 and heart rate 458 of the patient. These baseline values may represent values for pressure in the pulmonary artery, pressure in the right atrium, mean arterial pressure and the heart rate of the patient prior to undergoing a medical procedure.

Additionally, FIG. 21 illustrates that the display 400 may include a visual representation 450 of real-time values one or more physiological parameters and/or hemodynamic data (e.g., right arterial blood pressure, right ventricular pressure, cardiac output, stroke volume, etc.) of a patient. For example, FIG. 21 illustrates real-time values for the pressure in the pulmonary artery 460, pressure in the right atrium 462, mean arterial pressure 464 and heart rate 466 of the patient. These values may represent the actual, real-time values for pressure in the pulmonary artery, pressure in the right atrium, mean arterial pressure and the heart rate of the patient prior taken during a medical procedure.

FIG. 21 illustrates visual representations including baseline values for pressure in the pulmonary artery 452, pressure in the right atrium 454, mean arterial pressure 456 and heart rate 458 in addition to real-time values for pressure in the pulmonary artery 460, pressure in the right atrium 462, mean arterial pressure 464 and heart rate 466. However, this is not intended to be limiting. Rather, it is contemplated that the visual representations shown in the display 400 may include visual representations of a variety of physiological parameters including, but not limited to cardiac output, stroke volume, arterial pressure, venous pressure, superior vena cava pressure, inferior vena cava pressure, right atrial pressure, pulmonary artery pressure, mean arterial pressure, heart rate, renal artery pressure, renal vein pressure, combinations thereof or any other physiological parameter.

It can be further appreciated that, during a procedure, a user (e.g., a clinician) may compare the real-time values 460, 462, 464, 466 to the baseline parameters 452, 454, 456, 458 of the physiological parameters. Further the user may select, input and control various functionality of the medical device system 10 based on the comparison of the real-time values 460, 462, 464, 466 to the baseline parameters 452, 454, 456, 458 of the physiological parameters. For example, a user may select an “emergency stop” button 468 to immediately stop the inflation and/or deflation of the first and/or second expandable members on the comparison of the real-time values 460, 462, 464, 466 to the baseline parameters 452, 454, 456, 458 of the physiological parameters. In other examples, a user may utilize an “SVC Balloon” button 474 or an “IVC Balloon” button 476 in combination with an “Inflate” button 470 or “Deflate” button 472 and a “Volume to Dispense” selector 473 to inflate or deflate the first and/or second expandable members 430, 432 based on the comparison of the real-time values 460, 462, 464, 466 to the baseline parameters 452, 454, 456, 458 of the physiological parameters.

Additionally, the medical device system 10 (and/or other systems or components of medical systems disclosed herein) may collect cloud-based data of physiological parameters (e.g., blood pressure, cardiac output, stroke volume, arterial pressure, venous pressure, superior vena cava pressure, inferior vena cava pressure, right atrial pressure, pulmonary artery pressure, mean arterial pressure, heart rate, renal artery pressure, renal vein pressure), device sensor information, occlusion duty cycles, occlusion durations, occlusion location and use artificial intelligence or other advanced computational algorithms to optimize treatment for an individualized patient.

The materials that can be used for the various components of the system 10 (and/or other systems disclosed herein) may include those commonly associated with medical devices. However, this is not intended to limit the devices and methods described herein, as the discussion may be applied to other components, devices, or systems disclosed herein.

The components of the medical device system 10 (and/or other systems disclosed herein) may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the components of the system 10 (and/or other systems disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the components of the system 10 (and/or other systems disclosed herein) in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the components of the system 10 (and/or other systems disclosed herein) to achieve the same result.

In some embodiments, a degree of Magnetic Resonance Imaging (MRI) compatibility is imparted into the system 10 (and/or other systems disclosed herein). For example, components of the system 10 (and/or other systems disclosed herein), may be made of a material that does not substantially distort the image and create substantial artifacts (e.g., gaps in the image). Certain ferromagnetic materials, for example, may not be suitable because they may create artifacts in an MRI image. The components of the system 10 (and/or other systems disclosed herein) or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device system for treating a heart, the system comprising:
a control system including a processor and a pump;
a hub coupled to the control system;
a first catheter shaft having a first lumen and a first end coupled to the hub;
a second catheter shaft extending within the first lumen and having a first end couple to the hub;
a first expandable member disposed on the first catheter shaft, wherein the first expandable member is configured to be positioned in the superior vena cava; and
a second expandable member disposed on the second catheter shaft distal to the first expandable member, wherein the second expandable member is configured to translate relative to the first expandable member.

2. The medical device system of claim 1, wherein the second expandable member is configured to be positioned in the inferior vena cava.

3. The medical device system of claim 1, wherein the second expandable member is configured to be positioned adjacent to a renal artery.

4. The medical device system of claim 1, wherein the second expandable member is configured to translate relative to the first expandable member while the first expandable member is maintained in a fixed position within the superior vena cava.

5. The medical device system of claim 1, wherein the second expandable member is spaced apart from the first expandable member by a variable distance, and wherein the variable distances is 12 inches to 18 inches.

6. The medical device system of claim 1, wherein the first lumen is configured to permit an auxiliary medical device to pass through the first lumen into the right atrium of the heart.

7. The medical device system of claim 6, wherein the first lumen is configured to permit an auxiliary medical device to pass from the first lumen into the right atrium of the heart while the second expandable member is positioned in the inferior vena cava.

8. The medical device system of claim 6, wherein the pump is designed to expand or contract the first expandable member, the second expandable member, or both the first and the second expandable members based on a change in a first parameter sensed by the auxiliary medical device.

9. The medical device system of claim 8, wherein the first parameter is selected from the group consisting of a cardiac output of the heart, a blood flow in the pulmonary artery of the heart, and a blood pressure of the pulmonary artery of the heart.

10. The medical device system of claim 6, wherein the auxiliary medical device includes a thermodilution catheter.

11. The medical device of claim 8, further comprising a first sensing member having a first end positioned adjacent the first expandable member and a second end coupled to the control system, wherein the first sensing member is designed to sense a second parameter, and wherein the pump is designed to expand or contract the first expandable member based on a change in the first parameter, the second parameter, or a change in both the first and the second parameters.

12. The medical device of claim 8, further comprising a first sensing member having a first end positioned adjacent the first expandable member and a second end coupled to the control system, wherein the first sensing member is designed to sense a second parameter, and wherein the pump is designed to expand or contract the second expandable member based on a change in the first parameter, the second parameter, or a change in both the first and the second parameters.

13. The medical device of claim 11, further comprising a second sensing member having a first end positioned adjacent the second expandable member and a second end coupled to the control system, wherein the second sensing member is designed to sense a third parameter, and wherein the pump is designed to expand or contract the second expandable member based on a change in the first parameter, the second parameter, the third parameter, or a change in both the first and the second parameters, a change in both the first and the third parameters, or a change in both the second and the third parameters.

14. The medical device of claim 11, further comprising a second sensing member having a first end positioned adjacent the second expandable member and a second end coupled to the control system, wherein the second sensing member is designed to sense a third parameter, and wherein the pump is designed to expand or contract the first expandable member based on a change in the first parameter, the second parameter, the third parameter, or a change in both the first and the second parameters, a change in both the first and the third parameters, or a change in both the second and the third parameters.

15. The system of claim 13, wherein the second parameter, the third parameter, or both the second parameter and the third parameter is a blood pressure.

16. The system of claim 13, wherein the first sensing member, the second sensing member, or both the first sensing member and the second sensing member includes a pressure wire.

17. The system of claim 13, wherein the first sensing member, the second sensing member, or both the first sensing member and the second sensing member includes a fluid-filled sensing catheter.

18. A medical device system for treating a heart, the system comprising:
a control system including a processor and a pump;
a hub coupled to the control system;
a first catheter shaft having a first lumen and a first end coupled to the hub;
a second catheter shaft extending within the first lumen and having a first end coupled to the hub, wherein the second catheter shaft is configured to telescope relative to the first catheter shaft;
a first expandable member disposed on the first catheter shaft, wherein the first expandable member is configured to be positioned in the superior vena cava; and
a second expandable member disposed on the second catheter shaft distal to the first expandable member, wherein the second expandable member is configured to be positioned in the inferior vena cava while the first expandable member is held in a fixed position within the superior vena cava.

19. The medical device system of claim 18, wherein the first lumen is configured to permit an auxiliary medical device to pass through the first lumen into the right atrium of the heart.

20. A method for treating the heart, the method comprising:
advancing a medical device into the superior vena cava of the heart, the medical device including:
a first catheter shaft having a first lumen;
a second catheter shaft extending within the first lumen, wherein the second catheter shaft is configured to translate relative to the first catheter shaft;
a first expandable member disposed on the first catheter shaft; and
a second expandable member disposed on the second catheter shaft distal to the first expandable member;
a first sensing member having a first end positioned adjacent the first expandable member, the first sensing member designed to sense a first parameter;
a second sensing member having a first end positioned adjacent the second expandable member, the second sensing member designed to sense a second parameter;
positioning the first expandable member within the superior vena cava;
translating the second catheter shaft toward the inferior vena cava;
positioning the second expandable member within the inferior vena cava;
sensing a first parameter with the first sensing member;
sensing a second parameter with the second sensing member; and
expanding the first expandable member, the second expandable member or both the first and the second expandable members based on a change in the first parameter, a change in the second parameter, or a change in both the first and the second parameters.
